(19) United States
(12) Patent Application Publication
(10) Pub. No.: US 2026/0335522 A1
(43) Pub. Date: Oct. 29, 2026

(54) PERITONEAL DIALYSIS SYSTEM USING CYLINDER AND OPTIONALLY AIR PUMP

(71) Applicants: BAXTER INTERNATIONAL INC., Deerfield, IL (US); BAXTER HEALTHCARE SA, Glattpark (Opfikon) (CH)

(72) Inventor: BELUR Shanthakumar Shashanka Jain, Bangalore (IN)

(21) Appl. No.: 19/113,499

(22) PCT Filed: Oct. 5, 2023
(86) PCT No.: PCT/US2023/034565

§ 371 Date: Mar. 20, 2025

(30) Foreign Application Priority Data
Oct. 14, 2022 (IN) 202241058811

(51) Int. Cl. A61M 1/28; A61M 1/00; A61M 1/14

(52) U.S. Cl. CPC A61M 1/282; A61M 1/1565; A61M 1/71; A61M 2205/07

(57) ABSTRACT

In one example, a control unit causes (i) a second pneumatic valve and a source fluid valve to open and a linear actuator to move a piston head into a first cylinder chamber so as to create a negative pressure in a pneumatic pump chamber, pulling source fluid into a fluid pump chamber and (ii) a first pneumatic valve and a destination fluid valve to open and the linear actuator to move the piston head into the first cylinder chamber, pushing the source fluid through the destination fluid valve, the control unit using a pressure sensor reading to control the linear actuator so that a final pressure for (ii) at least substantially equals an initial pressure for (ii), such that a volume of space corresponding to the movement of piston head within the cylinder during (ii) equals a volume of the source fluid delivered from fluid pump chamber.

### PRIORITY CLAIM AND CROSS-REFERENCE TO RELATED APPLICATIONS

The present application is a national stage filing of PCT/US2023/034565, filed Oct. 5, 2023, which claims priority to Indian Patent App. No. 202241058811, filed Oct. 14, 2022, the entire contents of which are hereby incorporated by reference.

### BACKGROUND

The present disclosure relates generally to medical fluid treatments and in particular to dialysis fluid treatments.

Due to various causes, a person's renal system can fail. Renal failure produces several physiological derangements. It is no longer possible to balance water and minerals or to excrete daily metabolic load. Toxic end products of metabolism, such as, urea, creatinine, uric acid and others, may accumulate in a patient's blood and tissue.

Reduced kidney function and, above all, kidney failure is treated with dialysis. Dialysis removes waste, toxins and excess water from the body that normal functioning kidneys would otherwise remove. Dialysis treatment for replacement of kidney functions is critical to many people because the treatment is lifesaving.

One type of kidney failure therapy is Hemodialysis (“HD”), which in general uses diffusion to remove waste products from a patient's blood. A diffusive gradient occurs across the semi-permeable dialyzer between the blood and an electrolyte solution called dialysate or dialysis fluid to cause diffusion.

Hemofiltration (“HF”) is an alternative renal replacement therapy that relies on a convective transport of toxins from the patient's blood. HF is accomplished by adding substitution or replacement fluid to the extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi-or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days'worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, wherein the transfer of waste, toxins and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“PD”) is similar to CAPD in that the dialysis treatment includes drain, fill and dwell cycles. Automated PD machines, however, perform the cycles automatically, typically while the patient sleeps. Automated PD machines free patients from having to manually perform the treatment cycles and from having to transport supplies during the day. Automated PD machines connect fluidly to an implanted catheter, to a source or bag of fresh dialysis fluid and to a fluid drain. Automated PD machines pump fresh dialysis fluid from a dialysis fluid source, through the catheter and into the patient's peritoneal chamber. Automated PD machines also allow for the dialysis fluid to dwell within the chamber and for the transfer of waste, toxins and excess water to take place. The source may include multiple liters of dialysis fluid including several solution bags.

APD machines pump used or spent dialysate from the peritoneal chamber, though the catheter, and to the drain. As with the manual process, several drain, fill and dwell cycles occur during dialysis. A “last fill” may occur at the end of the APD treatment. The last fill fluid may remain in the peritoneal chamber of the patient until the start of the next treatment, or may be manually emptied at some point during the day.

In any of the above modalities using an automated machine, the automated machine operates typically with a disposable set, which is discarded after a single use. Depending upon the complexity of the disposable set, the cost of using one set per day may become significant. Also, daily disposables require space for storage, which can become a nuisance for home owners and businesses. Moreover, daily disposable replacement requires daily setup time and effort by the patient or caregiver at home or at a clinic.

There is also a need for APD devices to be portable so that a patient may bring his or her device on vacation or for work travel. Moreover, there is a need for APD devices to have pumping accuracy, so that the devices may accurately track how much ultrafiltration (“UF”) is removed from the patient over the course of treatment.

For each of the above reasons, it is desirable to provide a relatively simple, compact dialysis machine, such as an APD machine, which is accurate and operates a simple and cost effective disposable set.

### SUMMARY

The present disclosure relates to a peritoneal dialysis (“PD”) machine or cycler, which is driven by an air cylinder and optionally an air pump. In a first primary embodiment, only the air cylinder is provided. The air cylinder resides between first and second pneumatic pump chambers. A piston is located within the air cylinder, wherein the piston includes a piston head separating the air cylinder into a first cylinder chamber and a second cylinder chamber. A first pneumatic line extends from the first cylinder chamber to the first pneumatic pump chamber. A second pneumatic line extends from the second cylinder chamber to the second pneumatic pump chamber. A first pressure sensor is located so as to read air pressure in the first pneumatic line, the first pneumatic pump chamber and the first cylinder chamber. A second pressure sensor is located so as to read air pressure in the second pneumatic line, the second pneumatic pump chamber and the second cylinder chamber.

A first vent line and associated first vent valve are optionally placed in fluid communication with the first cylinder chamber. A second vent line and associated second vent valve are optionally placed in fluid communication with the second cylinder chamber. The piston further includes a piston shaft, which is coupled outside the air cylinder to a linear actuator for translating the piston shaft and piston head within the cylinder.

All valves, the motor of the linear actuator), a PD fluid heater and other controllable electrical devices are under control of a control unit, which includes at least one processor, at least one memory and a video controller for controlling a user interface. The control unit is further configured to receive signals from all sensors, such as pneumatic pressure sensors, fluid pressure sensors (if provided), a motor encoder (for the linear actuator if provided), and any temperature sensors associated with the heater. The control unit is programmed to run all pumping sequences discussed herein.

The PD machine or cycler operates with a disposable set in one embodiment. The disposable set among other items includes first and second fluid pump chambers that operate respectively with the first and second pneumatic pump chambers. When the piston head is moved so as to create a negative pneumatic pressure within the first or second cylinder chambers, a corresponding negative pressure is created in the respective first or second pneumatic pump chambers. The negative pressure created in the first or second pneumatic pump chamber in turn pulls a flexible membrane of the corresponding first or second fluid pump chamber into the first or second pneumatic pump chamber, such that the fluid pump chamber fills with fresh or used PD fluid. When the piston head is moved so as to create a positive pneumatic pressure within the first or second cylinder chambers, a corresponding positive pressure is created in the respective first or second pneumatic pump chambers. The positive pressure created in the first or second pneumatic pump chamber in turn pushes the flexible membrane of the corresponding first or second fluid pump chamber, such that the fluid pump chamber closes and expels fresh or used PD fluid.

The control unit in the first primary embodiment causes the piston shaft to translate the piston head back and forth within the air cylinder, such that in one half-stroke (i) the first pump chamber fills with fresh or used PD fluid, while the second fluid pump chamber expels fresh or used PD fluid. In a second half-stroke (ii) the second pump chamber fills with fresh or used PD fluid, while the first fluid pump chamber expels fresh or used PD fluid. The control unit causes the piston head to translate back and forth in the above-described manner until a desired or prescribed volume of fresh or used PD fluid from a desired PD fluid source is delivered to a desired PD fluid destination. Fluid valves are provided and are actuated sequentially by the control unit to access the desired fluid source and the desired fluid destination. The fluid valves may be such as magnetically actuated solenoid valves, motorized pinch valves, or pneumatically actuated valves.

In the first primary embodiment, and where patient pumping is taking place such that pressure control is important, the control unit monitors the outputs from the first and second pressure sensors while the piston head is translated back and forth. The control unit controls the speed of the back and forth translation such that a desirably safe negative or positive fluid pumping pressure is not exceeded.

In the first primary embodiment, the amount of fresh or used PD fluid delivered to a destination is determined by maintaining a constant pressure before and after movement of the piston head, which negates the effects of the compressibility of air within the cylinder. Because the pressure after movement (P2) equals the pressure before movement (P1), the volume of air within the cylinder remains constant. Hence, the volume displaced by the piston head is equal to volume of fluid delivered.

In a second primary embodiment, only the air cylinder is provided as before, but wherein the air cylinder is dedicated to a single pneumatic pump chamber/fluid pump chamber pair. Two pneumatic pump chamber/fluid pump chamber pairs may be provided, wherein each pair has its own dedicated air cylinder. The air cylinder is structured the same as in the first primary embodiment, including a piston head and piston shaft driven by a linear actuator. Optional vent lines and pneumatic vent valves may be pneumatically communicated with the first and second cylinder chambers of each air cylinder.

In the second primary embodiment, first and second pneumatic lines extend from the first and second cylinder chambers, respectively, to the same pneumatic pump chamber. First and second pneumatic valves under the control of control unit are provided along the first and second pneumatic lines. One or more pressure sensor is provided along a common portion of the first and second pneumatic lines or along each of the first and second pneumatic lines. The fluid pump chamber(s) is/are provided again as part of a disposable set, wherein the fluid pump chamber pumps fresh or used PD fluid from a desired PD fluid source to a desired destination as determined by the sequencing of one or more fluid valve.

The control unit in the second primary embodiment causes the piston shaft to translate the piston head towards one of the first or second cylinder chambers, e.g., the first cylinder chamber, creating positive pressure in the first cylinder chamber and negative pressure in the second cylinder chamber. The control unit also causes the source fluid valve and the second pneumatic valve to open, allowing negative pressure to reach the pneumatic pump chamber and causing the flexible membrane to be pulled into the pneumatic pump chamber and fill with fresh or used PD fluid.

Next, the control unit causes the source fluid valve and the second pneumatic valve to close and the first pneumatic valve to open so that the pressure sensor can read the positive pressure in the first cylinder chamber. The control unit also causes the piston to move into the first cylinder chamber such that the positive pressure in the pneumatic pump chamber reads a desired pressure, e.g., 1.5 psig, for pumping fresh or used PD fluid to the desired destination. At the end of such movement, the piston head is at an initial piston head position.

Next, the control unit maintains the first pneumatic valve in an open state and causes the destination fluid valve to open. The desired positive pressure built in the pneumatic pump chamber forces the flexible membrane of the fluid pump chamber to collapse and push fresh or used PD fluid to a desired destination. As the positive pressure dissipates, the control unit causes the piston to be moved further into the first cylinder chamber so that the pressure sensor continues to read the desired pressure, e.g., 1.5 psig.

Eventually, the flexible membrane cannot be collapsed any further, causing the pressure sensor reading to spike, at which time the control unit stops the pump-out translation of the piston head and closes the destination fluid valve. The detection of the flexible membrane not being able to collapse any further may be determined alternatively or additionally by the control unit detecting that the linear actuator and/or the piston head is/are not moving, while the desired pressure, e.g., 1.5 psig, is maintained. In any case, after stopping the pump-out translation, the first pneumatic valve remains open to allow the positive pressure, which has been maintained at the desired pressure, to equalize between the first cylinder chamber and the pneumatic pump chamber, and which may be read by the pressure sensor. The piston head is now at a final piston head position. The volume difference in the known cross-sectional area of the air cylinder between the final piston head position and the initial piston head position is the volume of fresh or used PD fluid pumped to the desired destination due to the pressures at the initial and final piston head positions being the same, e.g., 1.5 psig, which is a desirable pump-to-patient pressure. That is, a volume of space corresponding to the movement of the piston head within the cylinder is a function of a distance moved by the piston head within the cylinder and a cross-sectional area of an inner diameter of the cylinder.

Next, with the second cylinder chamber still being under negative pressure (which is not critical id pulling from a non-patient source), the control unit causes the source fluid valve and the second pneumatic valve to open, allowing the negative pressure to reach the pneumatic pump chamber and causing the flexible membrane to be pulled into the pneumatic pump chamber and fill with fresh or used PD fluid.

Next, the control unit closes the source fluid valve but allows the second pneumatic valve to remain open, such that the pneumatic pump chamber and the second cylinder chamber remain exposed to the pressure sensor. The control unit causes the piston to be translated into the second cylinder chamber until the pressure sensor reads zero psig. The pressure in the first cylinder chamber should also be close to zero psig.

Next, the control unit with the source and destination fluid valves closed, the first pneumatic valve closed, and the second pneumatic valve open so that the pressure sensor can read the positive pressure in the second cylinder chamber, causes the piston to move into the second cylinder chamber such that the positive pressure in the pneumatic pump chamber again reads a desired pressure, e.g., 1.5 psig, for pumping fresh or used PD fluid to the desired destination. At the end of such movement, the piston head is at again at an initial piston head position.

Next, the control unit maintains the second pneumatic valve in an open state and causes the destination fluid valve to open. The desired positive pressure built in the pneumatic pump chamber again forces the flexible membrane of the fluid pump chamber to collapse and push fresh or used PD fluid to the desired destination. As the positive pressure dissipates, the control unit causes the piston to be moved further into the second cylinder chamber so that the pressure sensor continues to read the desired pressure, e.g., 1.5 psig.

Eventually, the flexible membrane cannot be collapsed any further, causing the pressure sensor reading to spike, at which time the control unit stops the pump-out translation of the piston head and closes the destination fluid valve. The detection of the flexible membrane not being able to collapse any further may again be determined alternatively or additionally by the control unit detecting that the linear actuator and/or the piston head is/are not moving, while the desired pressure, e.g., 1.5 psig, is maintained. In any case, after stopping the pump-out translation, the second pneumatic valve remains open to allow the positive pressure, which has been maintained at the desired pressure, to equalize between the first cylinder chamber and the pneumatic pump chamber, and which may be read by the pressure sensor. The piston head is now at a final piston head position. The volume difference in the known cross-sectional area of the air cylinder between the final piston head position and the initial piston head position is again the volume of fresh or used PD fluid pumped to the desired destination due to the pressures at the initial and final piston head positions being the same, e.g., 1.5 psig, which is a desirable pump-to-patient pressure.

With the first cylinder chamber still being under negative pressure (which is not critical if pulling from a non-patient source), the control unit causes the source fluid valve and the first pneumatic valve to open, allowing the negative pressure to reach the pneumatic pump chamber and causing the flexible membrane to be pulled into the pneumatic pump chamber and fill with fresh or used PD fluid. The above process is repeated until a desired amount of fresh or used PD fluid is delivered to the desired destination. It should be appreciated that the above process may be used for any fresh or used PD fluid source and any fresh or used PD fluid destination described herein, and that both suction and delivery pressure and PD fluid volume delivered may be controlled and measured, respectively.

A third primary embodiment introduces an air pump that operates in cooperation with the air cylinder. Air pumps in general can transition more quickly between pumping positive versus negative pressure, and vice versa. Also, even small air pumps can create a wide range of pressures. Those two advantages of the air pump are combined with the air cylinder's ability to meter a known volume of fluid under pressure control as described herein.

The air cylinder in the third primary embodiment is structured roughly the same as in the first and second primary embodiments, and includes a piston head and piston shaft driven by a linear actuator. Optional vent lines and pneumatic vent valves may be pneumatically communicated with the first and second cylinder chambers of each air cylinder. In the third primary embodiment, only a first pneumatic line extends from the air cylinder to the pneumatic pump chamber. A first pneumatic valve under control of the control unit is provided along the first pneumatic line. A second pneumatic line extends from the air pump and meets with the first pneumatic line. A second pneumatic valve under control of the control unit is provided along the second pneumatic line. A pressure sensor is provided along a common portion of the first and second pneumatic lines. One or more fluid pump chamber is provided again as part of a disposable set, wherein the fluid pump chamber pumps fresh or used PD fluid from a desired PD fluid source to a desired destination as determined by the sequencing of one or more fluid valve.

The control unit in the third primary embodiment initially causes the first and second pneumatic valves to open, the source fluid valve to open and the air pump to create a negative pressure in the pneumatic pump chamber and the cylinder chamber, pulling the flexible membrane of the fluid pump chamber into the pneumatic pump chamber and fresh or used PD fluid into the fluid pump chamber. In an embodiment, the control unit during the PD fluid draw phase monitors a speed of the air pump. When the speed of the air pump begins to decrease, the control unit determines that the flexible membrane is fully pulled and expanded and therefore that the fluid pump chamber is full of fresh or used PD fluid. The speed of air pump decreases once the flexible membrane is fully pulled. The control unit provides closed loop control to the air pump so that the desired pressure is maintained. The control loop via the control unit ensures that pressure is not extending beyond a set threshold.

After the fluid pump chamber is fully filled with PD fluid, the control unit causes the source fluid valve to close. The control unit then causes the first and second pneumatic valves to open and the air pump to create a desired positive pumping pressure (e.g., 1.5 psig) in the pneumatic pump chamber and the cylinder chamber. Once the desired positive pumping pressure is reached, the control unit causes second pneumatic valve to close so that the air pump is isolated and blocked. The piston head of the piston is here at an initial piston head position.

The control unit next maintains the first pneumatic valve in an open state and causes the destination fluid valve to open. The desired positive pressure built in the pneumatic pump chamber forces the flexible membrane of the fluid pump chamber to collapse and push fresh or used PD fluid to the destination. As the positive pressure dissipates, the control unit causes the piston to be moved within the cylinder chamber so that the pressure sensor continues to read the desired pressure, e.g., 1.5 psig.

Eventually, the flexible membrane cannot be collapsed any further, causing the pressure sensor reading to spike, at which time the control unit stops the pump-out translation of the piston head and closes the destination fluid valve. The detection of the flexible membrane not being able to collapse any further may again be determined alternatively or additionally by the control unit detecting that the linear actuator and/or the piston head is/are not moving, while the desired pressure, e.g., 1.5 psig, is maintained. In any case, after stopping the pump-out translation, the first pneumatic valve remains open to allow the positive pressure, which has been maintained at the desired pressure, to equalize between the cylinder chamber and the pneumatic pump chamber, and which may be read by the pressure sensor. The piston head is now at a final piston head position. The volume difference in the known cross-sectional area of the air cylinder between the final piston head position and the initial piston head position is the volume of fresh PD fluid pumped to the destination due to the pressures at the initial and final piston head positions being the same, e.g., 1.5 psig, which is a desirable pump-to-patient pressure.

The control unit then causes the first and second pneumatic valves to open, the source fluid valve to open and the piston to be moved in the opposite direction within the cylinder to reposition the piston head for the next pump-out stroke. Movement of the piston creates negative pressure within the cylinder chamber and the pneumatic pump chamber, which may be aided by the air pump to quickly achieve a desired PD fluid draw pressure. The flexible membrane of the fluid pump chamber is pulled into the pneumatic pump chamber and fresh or used PD fluid is pulled correspondingly into the fluid pump chamber. The above process for the third primary embodiment is repeated, wherein the control unit accumulates the pump stroke volumes, until a desired or prescribed amount of fresh or used PD fluid is delivered to the destination.

A fourth primary embodiment also uses an air pump that operates in cooperation with the air cylinder. Here, a single air pump and air cylinder are able to drive two fluid pump chambers within two pneumatic pump chambers. The air cylinder in the fourth primary embodiment is structured the same as in the third primary embodiment, and includes a piston head and piston shaft driven by a linear actuator. Optional vent lines and pneumatic vent valves may be pneumatically communicated with the first and second cylinder chambers of each air cylinder. In the fourth primary embodiment, only a first pneumatic line extends from the air cylinder, however, the first pneumatic line splits to also include a second pneumatic line, wherein the first and second pneumatic lines extend respectively to first and second pneumatic pump chambers. First and second pneumatic valves under control of the control unit are provided along the first and second pneumatic lines, respectively.

A third pneumatic line extends from the air pump and splits into a fourth pneumatic line. The third pneumatic line meets with the first pneumatic line, while the fourth pneumatic line meets with the second pneumatic line. A third pneumatic valve under control of the control unit is provided along the third pneumatic line, while a fourth pneumatic valve under control of the control unit is provided along the fourth pneumatic line. A first pressure sensor is provided adjacent to the first pneumatic pump chamber, while a second pressure sensor is provided adjacent to the second pneumatic pump chamber. First and second fluid pump chambers are provided as part of a disposable set, wherein the first and second fluid pump chambers pump fresh or used PD fluid from a desired PD fluid source to a desired PD fluid destination as determined by the sequencing of a plurality of fluid valves.

In a pumping sequence for the fourth primary embodiment, the first and second fluid pump chambers are generally alternated, wherein as one fluid pump chamber draws fresh or used PD fluid in, the other fluid pump chamber pushes fresh or used PD fluid out. Each fluid pump chamber has its own set of source and destination valves, however, it is not required that the first and second fluid pump chambers are perfectly synched.

The control unit in the fourth primary embodiment initially causes the third pneumatic valve and the source fluid valve for the first fluid pump chamber to open and the air pump to create a negative pressure in the first pneumatic pump chamber, pulling the flexible membrane of the first fluid pump chamber into the first pneumatic pump chamber and fresh or used PD fluid into the first fluid pump chamber. The control unit during the PD fluid draw phase may again monitor a speed of the air pump. When the speed of the air pump begins to decrease beyond a set threshold, the control unit determines that the flexible membrane is fully pulled and expanded and therefore that the fluid pump chamber is full of fresh or used PD fluid, at which time the control unit causes the air pump to stop and the source fluid valve for the first fluid pump chamber to close. The control unit next maintains the third pneumatic valve in an open state and causes the air pump to create a desired positive pumping pressure (e.g., 1.5 psig as read by the first pressure sensor) in the first pneumatic pump chamber. At this point, the piston head of the piston of the air cylinder is at an initial piston head position.

The control unit next causes the third pneumatic valve to close, the first pneumatic valve to open and the first destination fluid valve to open. The desired positive pressure built in the first pneumatic pump chamber forces the flexible membrane of the first fluid pump chamber to collapse and push fresh or used PD fluid to the destination. As the positive pressure dissipates, the control unit causes the piston to be moved within the cylinder chamber so that the first pressure sensor continues to read the desired pressure, e.g., 1.5 psig. Simultaneously, the control unit causes the fourth pneumatic valve to open, the second source valve to open, and the air pump to create a negative pressure in the second pneumatic pump chamber, pulling the flexible membrane of the second fluid pump chamber into the second pneumatic pump chamber and fresh or used PD fluid into the second fluid pump chamber.

Eventually, the first flexible membrane cannot be collapsed any further, causing the first pressure sensor reading to spike, at which time the control unit stops the pump-out translation of the piston head and closes the first destination fluid valve. The detection of the flexible membrane not being able to collapse any further may be determined alternatively or additionally by the control unit detecting that the linear actuator and/or the piston head is/are not moving, while the desired pressure, e.g., 1.5 psig, is maintained. In any case, after stopping the pump-out translation, the first pneumatic valve remains open to allow the positive pressure, which has been maintained at the desired pressure, to equalize between the cylinder chamber and the first pneumatic pump chamber, and which may be read by the first pressure sensor. The piston head is now at a final piston head position within the cylinder chamber. The volume difference in the known cross-sectional area of the air cylinder between the final piston head position and the initial piston head position is the volume of fresh or used PD fluid pumped to the destination due to the pressures at the initial and final piston head positions being the same, e.g., 1.5 psig, which is a desirable pump-to-patient pressure. For the fluid draw of the second fluid pump chamber, the control unit may again monitor a speed of the air pump. When the speed of the air pump begins to decrease beyond a set threshold, the control unit determines that the flexible membrane of the second fluid pump chamber is fully pulled and expanded and therefore that the second fluid pump chamber is full of fresh or used PD fluid, at which time the control unit causes the air pump to stop and the second source fluid valve for the second fluid pump chamber to close.

The control unit then causes the piston to retract to an initial position and the above process to be repeated, but wherein the first fluid pump chamber fills with fresh or used PD fluid and the second fluid pump chamber pushes fresh or used PD fluid to the destination. The control unit accumulates the known stroke volumes and continues to perform the alternating pumping sequence just described until a desired or prescribed amount of fresh or used PD fluid is delivered to the destination.

A fifth primary embodiment, like the fourth primary embodiment, also uses an air pump that operates in cooperation with the air cylinder to drive two pneumatic pump chambers and corresponding fluid pump chambers. In the fourth primary embodiment, the air cylinder is unidirectional regarding fluid volume metering because only one side of the piston head within the cylinder is exposed to the first and second pressure sensors. The piston needs to be reset accordingly after each fluid pump chamber draw/fluid pump chamber deliver sequence. In the fifth primary embodiment, a fifth pneumatic line is added, which extends from the first pneumatic line to an opposing side of the air cylinder, so that there is pneumatic access to the air cylinder on both sides of the piston head. A fifth pneumatic valve is provided with the fifth pneumatic line. A sixth pneumatic valve is added as a second valve along the first pneumatic line, which allows the air cylinder on that side of the piston head to be closed-off.

In a pumping sequence using the fifth primary embodiment, the control unit may cause the second and fifth pneumatic valves to be open, the second destination fluid valve to be open, and the piston head of the air cylinder to be moved in a first direction to deliver fresh or used PD fluid from the second fluid pump chamber to a desired destination. Simultaneously, the control unit causes the third pneumatic valve and the first source fluid valve to be open so that the air pump may apply a negative pressure to the flexible membrane of the first fluid pump chamber, pulling fresh or used PD fluid into the first fluid pump chamber.

Eventually, the second flexible membrane cannot be collapsed any further, causing the second pressure sensor reading to spike, at which time the control unit stops the pump-out translation of the piston head and closes the second destination fluid valve. The detection of the flexible membrane not being able to collapse any further may again be determined alternatively or additionally by the control unit detecting that the linear actuator and/or the piston head is/are not moving, while the desired pressure, e.g., 1.5 psig, is maintained. In any case, after stopping the pump-out translation, the second and fifth pneumatic valves remain open to allow the positive pressure, which has been maintained at the desired pressure, to equalize between the cylinder chamber and the second pneumatic pump chamber, and which may be read by the second pressure sensor. The volume difference in the known cross-sectional area of the air cylinder between the initial and final piston head positions is the volume of fresh or used PD fluid pumped to the destination due to the pressures at the initial and final piston head positions being the same, e.g., 1.5 psig, which is a desirable pump-to-patient pressure. For the fluid draw of the first fluid pump chamber, the control unit may again monitor a speed of the air pump. When the speed of the air pump begins to decrease beyond a set threshold, the control unit determines that the flexible membrane of the first fluid pump chamber is fully pulled and expanded and therefore that the first fluid pump chamber is full of fresh or used PD fluid, at which time the control unit causes the air pump to stop and the first source fluid valve for the first fluid pump chamber to close.

Next, the first and second fluid pump chambers switch operation so that the second fluid pump chamber draws fresh or used PD fluid in, while the first fluid pump chamber delivers the fresh or used PD fluid. Notably, no adjustment of the piston head is needed to ready the air cylinder for the switch. Here, the control unit causes the first and sixth pneumatic valves to be open, the first destination fluid valve to be open and the piston head of the air cylinder to be moved in a second direction to deliver fresh or used PD fluid from the first fluid pump chamber to the desired destination. Simultaneously, the control unit causes the fourth pneumatic valve and the second source fluid valve to be open so that the air pump may apply a negative pressure to the flexible membrane of the second fluid pump chamber, pulling fresh or used PD fluid into the second fluid pump chamber.

Eventually, the first flexible membrane cannot be collapsed any further, causing the first pressure sensor reading to spike, at which time the control unit stops the pump-out translation of the piston head and closes the first destination fluid valve. The detection of the flexible membrane not being able to collapse any further may again be determined alternatively or additionally by the control unit detecting that the linear actuator and/or the piston head is/are not moving, while the desired pressure, e.g., 1.5 psig, is maintained. In any case, after stopping the pump-out translation, the first and sixth pneumatic valves remain open to allow the positive pressure, which has been maintained at the desired pressure, to equalize between the cylinder chamber and the first pneumatic pump chamber, and which may be read by the first pressure sensor. The volume difference in the known cross-sectional area of the air cylinder between the initial and final piston head positions is the volume of fresh or used PD fluid pumped to the destination due to the pressures at the initial and final piston head positions being the same, e.g., 1.5 psig, which is a desirable pump-to-patient pressure. For the fluid draw of the second fluid pump chamber, the control unit may again monitor a speed of the air pump. When the speed of the air pump begins to decrease beyond a set threshold, the control unit determines that the flexible membrane of the second fluid pump chamber is fully pulled and expanded and therefore that the second fluid pump chamber is full of fresh or used PD fluid, at which time the control unit causes the air pump to stop and the second source fluid valve for the second fluid pump chamber to close.

The control unit accumulates the known stroke volumes and continues to perform the alternating pumping sequence just described until a desired or prescribed amount of fresh or used PD fluid is delivered to the destination. It should be appreciated for any of the above primary embodiments that for fresh or used PD fluid destinations that do not involve the patient (e.g., heater bag or drain), the delivery pressure may be higher, e.g., up to eight psig. It is also contemplated for any of the primary embodiments that the control unit monitors the relevant first or second pressure sensor when either the air cylinder or the air pump is removing used PD fluid from the patient, so that a patient drain negative pressure limit, e.g., −1.5 psig, is not met or is not exceeded.

It should be appreciated that the effects of drift in the pneumatic pressure sensors for the above embodiments are negated because the important aspect for the above air cylinder sequences is that the initial and final pressures associated with fresh or used PD fluid delivery are equal, and not that the pressures are accurate from an absolute standpoint (except for patient pressure limits). Also, because the system is pressure controlled, the linear actuator does not have to be highly accurate.

In light of the disclosure set forth herein, and without limiting the disclosure in any way, in a first aspect of the present disclosure, which may be combined with any other aspect or portion thereof, a peritoneal dialysis system includes a pneumatic pump chamber; a cylinder; a piston including a piston head slideably sealed within the cylinder, the piston head separating a first cylinder chamber from a second cylinder chamber; a linear actuator in mechanical communication with the piston; a first pneumatic line extending between the first cylinder chamber and the pneumatic pump chamber; a second pneumatic line extending between the second cylinder chamber and the pneumatic pump chamber; a first pneumatic valve located along the first pneumatic line; a second pneumatic valve located along the second pneumatic line; a pressure sensor positioned and arranged to measure a pressure in the pneumatic pump chamber; a fluid pump chamber operably coupled with the pneumatic pump chamber; a source fluid valve; a destination fluid valve; and a control unit configured to cause (i) the second pneumatic valve and the source fluid valve to open and the linear actuator to move the piston head into the first cylinder chamber so as to create a negative pneumatic pressure in the second cylinder chamber and the pneumatic pump chamber, pulling source fluid into the fluid pump chamber and (ii) the second pneumatic valve and the source fluid valve to close, the first pneumatic valve and the destination fluid valve to open and the linear actuator to move the piston head further into the first cylinder chamber so as to push source fluid through the destination fluid valve, and wherein the control unit uses an output from the pressure sensor to control the linear actuator so that a final pressure for (ii) at least substantially equals an initial pressure for (ii), such that a volume of space corresponding to the movement of the piston head within the cylinder during (ii) equals a volume of the source fluid delivered from fluid pump chamber during (ii).

In a second aspect of the present disclosure, which may be combined with any other aspect or portion thereof, the source fluid valve is for a PD fluid supply container, a heating container, or a patient line.

In a third aspect of the present disclosure, which may be combined with any other aspect or portion thereof, the destination fluid valve is for a heating container, a drain container, or a patient line.

In a fourth aspect of the present disclosure, which may be combined with any other aspect or portion thereof, the volume of space corresponding to the movement of the piston head within the cylinder is a function of a distance moved by the piston head within the cylinder and a cross-sectional area of an inner diameter of the cylinder.

In a fifth aspect of the present disclosure, which may be combined with any other aspect or portion thereof, the control unit is configured to determine the volume of the source fluid delivered from fluid pump chamber during (ii) with both the source fluid valve and the destination fluid valve closed.

In a sixth aspect of the present disclosure, which may be combined with any other aspect or portion thereof, the control unit is further configured to cause, with the first pneumatic valve and the destination fluid valve closed, (iii) the second pneumatic valve and the source fluid valve to open to allow the negative pneumatic pressure in the second cylinder chamber created during at least one of (i) or (ii) to reach the pneumatic pump chamber, pulling source fluid into the fluid pump chamber.

In a seventh aspect of the present disclosure, which may be combined with any other aspect or portion thereof, the control unit is further configured to cause, with the first pneumatic valve, the source fluid valve and the destination fluid valve closed and the second pneumatic valve closed, (iv) the linear actuator to move the piston head into the second cylinder chamber so as to create at least substantially zero pressure in the first and second cylinder chambers.

In an eighth aspect of the present disclosure, which may be combined with any other aspect or portion thereof, the control unit is further configured to cause (v) the second pneumatic valve and the destination fluid valve to open and the linear actuator to move the piston head into the second cylinder chamber so as to push source fluid through the destination fluid valve, and wherein the control unit uses an output from the pressure sensor to control the linear actuator so that a final pressure for (v) at least substantially equals an initial pressure for (v), such that a volume of space corresponding to the movement of the piston head within the cylinder during (v) equals a volume of the source fluid delivered from fluid pump chamber during (v).

In a ninth aspect of the present disclosure, which may be combined with any other aspect or portion thereof, the control unit is further configured to cause, with the second pneumatic valve and the destination fluid valve closed, (vi) the first pneumatic valve and the source fluid valve to open to allow the negative pneumatic pressure in the first cylinder chamber created during (v) to reach the pneumatic pump chamber, pulling source fluid into the fluid pump chamber.

In a tenth aspect of the present disclosure, which may be combined with any other aspect or portion thereof, a peritoneal dialysis system includes a pneumatic pump chamber; a cylinder; a piston including a piston head slideably sealed within the cylinder; a linear actuator in mechanical communication with the piston; an air pump; a first pneumatic line extending between the cylinder and the pneumatic pump chamber; a second pneumatic line extending between the air pump and the pneumatic pump chamber; a first pneumatic valve located along the first pneumatic line; a second pneumatic valve located along the second pneumatic line; a pressure sensor positioned and arranged to measure a pressure in the pneumatic pump chamber; a fluid pump chamber operably coupled with the pneumatic pump chamber; a source fluid valve; a destination fluid valve; and a control unit configured to cause (i) the second pneumatic valve and the source fluid valve to open and the air pump to create a negative pneumatic pressure in the pneumatic pump chamber, pulling source fluid into the fluid pump chamber, (ii) the source fluid valve to close and with the second pneumatic valve open, the air pump to create a desired positive pressure, as measured by the pressure sensor, in the pneumatic pump chamber, and (iii) the second pneumatic valve to close, the first pneumatic valve and the destination fluid valve to open, and the linear actuator to move the piston head within the cylinder so as to push source fluid through the destination fluid valve, and wherein the control unit uses an output from the pressure sensor to control the linear actuator so that a final pressure for (iii) at least substantially equals an initial pressure for (iii), such that a volume of space corresponding to the movement of the piston head within the cylinder during (iii) equals a volume of the source fluid delivered from fluid pump chamber during (iii).

In an eleventh aspect of the present disclosure, which may be combined with any other aspect or portion thereof, the source fluid valve is for a PD fluid supply container, a heating container, or a patient line.

In a twelfth aspect of the present disclosure, which may be combined with any other aspect or portion thereof, the destination fluid valve is for a heating container, a drain container, or a patient line.

In a thirteenth aspect of the present disclosure, which may be combined with any other aspect or portion thereof, the volume of space corresponding to the movement of the piston head within the cylinder is a function of a distance moved by the piston head within the cylinder and a cross-sectional area of an inner diameter of the cylinder.

In a fourteenth aspect of the present disclosure, which may be combined with any other aspect or portion thereof, during (i) the first pneumatic valve is open.

In a fifteenth aspect of the present disclosure, which may be combined with any other aspect or portion thereof, the control unit is further configured to cause, with the first pneumatic valve and the source fluid valve open, (iv) the linear actuator to move the piston head within the cylinder in an opposite direction, pulling source fluid into the fluid pump chamber.

In a sixteenth aspect of the present disclosure, which may be combined with any other aspect or portion thereof, during (iv) the first pneumatic valve is open and the air pump is actuated to aid in pulling source fluid into the fluid pump chamber.

In a seventeenth aspect of the present disclosure, which may be combined with any other aspect or portion thereof, a peritoneal dialysis system includes a first pneumatic pump chamber; a second pneumatic pump chamber; a cylinder; a piston including a piston head slideably sealed within the cylinder; a linear actuator in mechanical communication with the piston; an air pump; a first pneumatic line extending between the cylinder and the first pneumatic pump chamber; a second pneumatic line extending between the cylinder and the second pneumatic pump chamber; a third pneumatic line extending between the air pump and the first pneumatic pump chamber; a fourth pneumatic line extending between the air pump and the second pneumatic pump chamber; a first pneumatic valve located along the first pneumatic line; a second pneumatic valve located along the second pneumatic line; a third pneumatic valve located along third first pneumatic line; a fourth pneumatic valve located along the fourth pneumatic line; a first pressure sensor positioned and arranged to measure a pressure in the first pneumatic pump chamber; a second pressure sensor positioned and arranged to measure a pressure in the second pneumatic pump chamber; a first fluid pump chamber operably coupled with the first pneumatic pump chamber; a first source fluid valve for the first pump chamber; a first destination fluid valve for the first pump chamber; a second fluid pump chamber operably coupled with the first pneumatic pump chamber; a second source fluid valve for the second pump chamber; a second destination fluid valve for the second pump chamber; and a control unit configured to use the air pump to create negative and positive pneumatic pressures in the first and second pneumatic pump chambers, and to actuate the linear actuator to move the piston head within the cylinder while equalizing initial and final positive pneumatic pressures to meter determinable volumes of source fluid through the first and second destination fluid valves.

In an eighteenth aspect of the present disclosure, which may be combined with any other aspect or portion thereof, the control unit is configured to cause: (i) with the third pneumatic valve and the first source fluid valve open, the air pump to create a negative pneumatic pressure in the first pneumatic pump chamber, pulling source fluid into the first fluid pump chamber, (ii) with the third pneumatic valve open and the first source fluid valve closed, the air pump to create a desired positive pneumatic pressure in the first pneumatic pump chamber as measured by the first pressure sensor, and (iii) with the first pneumatic valve and the first destination fluid valve open, the linear actuator to move the piston head within the cylinder so as to push source fluid through the first destination fluid valve, and wherein the control unit uses an output from the first pressure sensor to control the linear actuator so that a final pressure for (iii) at least substantially equals an initial pressure for (iii), such that a volume of space corresponding to the movement of the piston head within the cylinder during (iii) equals a volume of the source fluid delivered from the first fluid pump chamber during (iii), and with the fourth pneumatic valve and the second source fluid valve open, the air pump to create a negative pneumatic pressure in the second pneumatic pump chamber, pulling source fluid into the second fluid pump chamber.

In a nineteenth aspect of the present disclosure, which may be combined with any other aspect or portion thereof, the control unit is further configured to cause, with the piston head moved to a retracted position: (iv) with the fourth pneumatic valve open and the second source fluid valve closed, the air pump to create a desired positive pneumatic pressure in the second pneumatic pump chamber as measured by the first pressure sensor, and (v) with the second pneumatic valve and the second destination fluid valve open, the linear actuator to move the piston head within the cylinder so as to push source fluid through the second destination fluid valve, and wherein the control unit uses an output from the second pressure sensor to control the linear actuator so that a final pressure for (v) at least substantially equals an initial pressure for (v), such that a volume of space corresponding to the movement of the piston head within the cylinder during (v) equals a volume of the source fluid delivered from the second fluid pump chamber during (v), and with the third pneumatic valve and the first source fluid valve open, the air pump to create a negative pneumatic pressure in the first pneumatic pump chamber, pulling source fluid into the first fluid pump chamber.

In a twentieth aspect of the present disclosure, which may be combined with any other aspect or portion thereof, a peritoneal dialysis system includes a first pneumatic pump chamber; a second pneumatic pump chamber; a cylinder; a piston including a piston head slideably sealed within the cylinder, the piston head separating a first cylinder chamber from a second cylinder chamber; a linear actuator in mechanical communication with the piston; an air pump; a first pneumatic line extending between the second cylinder chamber and the first pneumatic pump chamber; a second pneumatic line extending between the second cylinder chamber and the second pneumatic pump chamber; a third pneumatic line extending between the air pump and the first pneumatic pump chamber; a fourth pneumatic line extending between the air pump and the second pneumatic pump chamber; a fifth pneumatic line extending between the first cylinder chamber and the first pneumatic line; a first pneumatic valve located along the first pneumatic line; a second pneumatic valve located along the second pneumatic line; a third pneumatic valve located along third first pneumatic line; a fourth pneumatic valve located along the fourth pneumatic line; a fifth pneumatic valve located along the fifth pneumatic line; a sixth pneumatic valve located adjacent to the second cylinder chamber; a first pressure sensor positioned and arranged to measure a pressure in the first pneumatic pump chamber; a second pressure sensor positioned and arranged to measure a pressure in the second pneumatic pump chamber; a first fluid pump chamber operably coupled with the first pneumatic pump chamber; a first source fluid valve for the first pump chamber; a first destination fluid valve for the first pump chamber; a second fluid pump chamber operably coupled with the first pneumatic pump chamber; a second source fluid valve for the second pump chamber; a second destination fluid valve for the second pump chamber; and a control unit configured to use the air pump to create negative and positive pneumatic pressures in the first and second pneumatic pump chambers, and to actuate the linear actuator to move the piston head within the first and second cylinder chambers while equalizing initial and final positive pneumatic pressures to meter determinable volumes of source fluid through the first and second destination fluid valves.

In a twenty-first aspect of the present disclosure, which may be combined with any other aspect or portion thereof, the control unit is configured to cause (i) with the third pneumatic valve and the first source fluid valve open, the air pump to create a negative pneumatic pressure in the first pneumatic pump chamber, pulling source fluid into the first fluid pump chamber, and with the second and fifth pneumatic valves and the second destination fluid valve open, the linear actuator to move the piston head towards the first cylinder chamber so as to push source fluid through the second destination fluid valve, and wherein the control unit uses an output from the second pressure sensor to control the linear actuator so that a final pressure for (i) at least substantially equals an initial pressure for (i), such that a volume of space corresponding to the movement of the piston head towards the first cylinder chamber during (i) equals a volume of the source fluid delivered from the second fluid pump chamber during (i).

In a twenty-second aspect of the present disclosure, which may be combined with any other aspect or portion thereof, the control unit is further configured to cause (ii) with the fourth pneumatic valve and the second source fluid valve open, the air pump to create a negative pneumatic pressure in the second pneumatic pump chamber, pulling source fluid into the second fluid pump chamber, and with the first and sixth pneumatic valves and the first destination fluid valve open, the linear actuator to move the piston head towards the second cylinder chamber so as to push source fluid through the first destination fluid valve, and wherein the control unit uses an output from the first pressure sensor to control the linear actuator so that a final pressure for (ii) at least substantially equals an initial pressure for (ii), such that a volume of space corresponding to the movement of the piston head towards the second cylinder chamber during (ii) equals a volume of the source fluid delivered from the second fluid pump chamber during (ii).

In a twenty-third aspect, which may be combined with any other aspect or portion thereof, any of the features, functionality and alternatives described in connection with any one or more of FIGS. 1 to 28 may be combined with any of the features, functionality and alternatives described in connection with any other of FIGS. 1 to 28.

It is accordingly an advantage of the present disclosure to provide a relatively volumetrically accurate automated peritoneal dialysis (“PD”) machine.

It is another advantage of the present disclosure to provide a PD machine that achieves relatively precise pressure control.

It is a further advantage of the present disclosure to provide a relatively quiet PD machine.

It is still another advantage of the present disclosure to provide an PD machine that is accurate irrespective of drift in a pressure sensor with time, temperature, humidity, etc.

It is yet another advantage of the present disclosure to provide a PD machine that removes a dependence of volumetric accuracy on absolute pressure sensing.

It is yet a further advantage of the present disclosure to provide a PD system that uses both a low cost and simple machine and a low cost and simple disposable.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic elevation view of one embodiment for a peritoneal dialysis (“PD”) system of the resent disclosure.

FIGS. 2 and 3 are schematic views of a first primary embodiment for the PD system of the present disclosure, which uses an air cylinder.

FIGS. 4 to 13 are schematic views of a second primary embodiment for the PD system of the present disclosure, which uses an air cylinder.

FIGS. 14 to 20 are schematic views of a third primary embodiment for the PD system of the present disclosure, which uses an air cylinder and an air pump.

FIGS. 21 to 24 are schematic views of a fourth primary embodiment for the PD system of the present disclosure, which uses an air cylinder and an air pump.

FIGS. 25 to 28 are schematic views of a fifth primary embodiment for the PD system of the present disclosure, which uses an air cylinder and an air pump.

### DETAILED DESCRIPTION

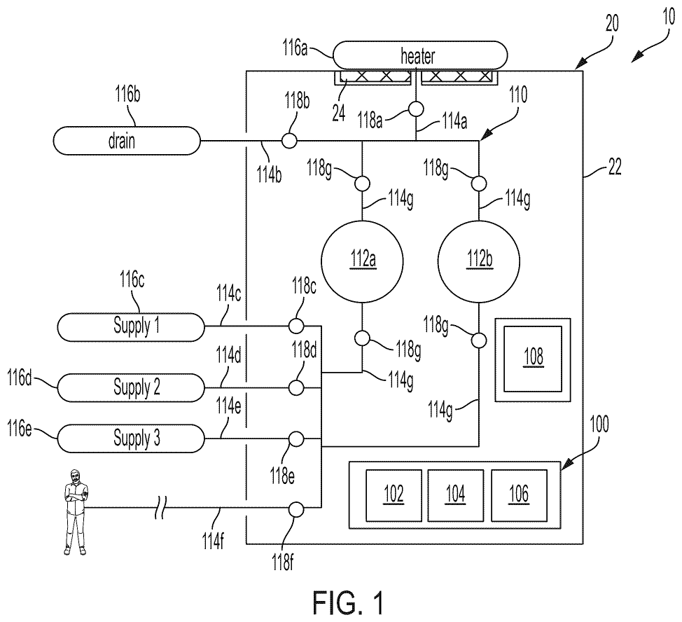

Referring now to the drawings and in particular to FIG. 1, an automated peritoneal dialysis (“PD”) system 10 includes a PD machine or cycler 20 that operates with a disposable set 110. Disposable set 110 includes or defines at least one fluid pump chamber 112a, 112b, which may include two flexible sheets welded together to form a, e.g., circular chamber that can be spread apart in a spherical manner. At least one fluid pump chamber 112a, 112b may alternatively include a single flexible sheet welded to a rigid, semi-spherical pump shell. All welding discussed herein may be via heat sealing, ultrasonic sealing or solvent bonding.

Disposable set 110 also includes or defines a plurality of fresh and used PD fluid lines, such as heater line 114a, drain line 114b, PD fluid supply lines 114c, 114 d, 114e, patient line 114f and fluid pump chamber lines 114g. Fresh and used PD fluid lines may be formed via tubing, via welding pathways between two flexible sheets, or via molding pathways in a rigid cassette.

Disposable set 110 further includes a plurality of PD fluid containers, such as a heating container 116a, a drain container 116b, PD fluid supply containers 116c, 116d, and 116e. Heating container 116a in the illustrated embodiment is located on a heating tray at the top of a housing 22 of PD machine or cycler 20. A batch heater 24, such as an electrical resistance heater, under control of a control unit 100 is provided at the top of PD machine or cycler 20. In an alternative embodiment, an initial one of supply containers 116c, 116d, and 116e is placed on the heating tray and then once empty is used as the heating container for the rest of treatment. In a further alternative embodiment, heater 24 is instead an inline heater, e.g., operating with patient line 114f, such that heating container 116a may be eliminated. In any case, control unit 100 is programmed to cause heater 24 to heat fresh PD fluid to a patient temperature, e.g., 37° C.

Any desired number and sizes of PD fluid supply containers 116c, 116d, 116e may be provided and may hold the same or different dextrose or glucose level PD fluids. One of PD fluid supply containers 116c, 116d, 116e may be a last fill container and contain a different PD formulation, such as icodextrin. Additionally, drain line 114b may lead instead to a house drain, such as a toilet or bathtub, in which case drain container 116b is not needed. Any of containers 116a to 116a may be formed as a flexible container or bag.

Disposable set 110 in the illustrated embodiment further includes a plurality of fluid valve seats, such as, heater valve seat 118a, drain valve seat 118b, PD fluid supply valve seats 118c, 118d, 118e, patient valve seat 118f and fluid pump chamber valve seats 118g. Fluid valve seats 118a to 118g may be tubing locations, formed via welding two flexible sheets, or via molding in a rigid cassette. Fluid valve seats 118a to 118g operate with valve actuators (not seen in FIG. 1), which may be magnetically actuated solenoid pinch valve actuators, motorized pinch valves, or pneumatically actuated valve actuators.

In system 10, and as described in detail below, supply containers 116c, 116d, and 116e are fresh PD fluid sources ((“FS”) although one could be a fresh PD fluid destination if used as a heating container). Drain container 116b is a used PD fluid destination (“FD”). Heating container 116a is a fresh PD fluid source (“FS”) and a fresh PD fluid destination (“FD”). Patient line 114f (the patient) is a used PD fluid source (“FS”) and a fresh PD fluid destination (“FD”).

In system 10, and as described in detail below, PD fluid supply valve seats 118c, 118d, and 118e are source fluid valves ((“SV”) although one could be a destination fluid valve (“DV”) if used as heater valve). Drain valve seat 118b is a destination fluid valve (“DV”). Heater valve seat 118a and patient valve seat are source fluid valves (“SV”) and destination fluid valves (“DV”). Fluid pump chamber valve seats 118g allow fluid pump chambers 112a, 112b to alternate drawing fresh or used PD fluid in or pumping fresh or used PD fluid out, depending on the current pumping sequence, so that flow the desired destination is relatively continuous.

Any rigid component of disposable set 110 may be made of plastic, such as, polyvinyl chloride (“PVC”), polyethylene (“PE”), polyurethane (“PU”) or polycarbonate (“PC”). Any flexible components of disposable set 110, such as membranes or diaphragms, tubing and the containers discussed herein may be made of a medically safe material such as one or more plastic, e.g., PVC, PE, PU, or other suitable non-PVC polymer. Rigid pump housing 22, the air cylinder and its associated components discussed herein, the pneumatic lines discussed herein, which are reusable in one embodiment and may be made of plastic, such as, polyvinyl chloride (“PVC”), polyethylene (“PE”) or polyurethane (“PU”), or of metal, such as stainless steel or aluminum, and combinations thereof.

### First Primary Embodiment

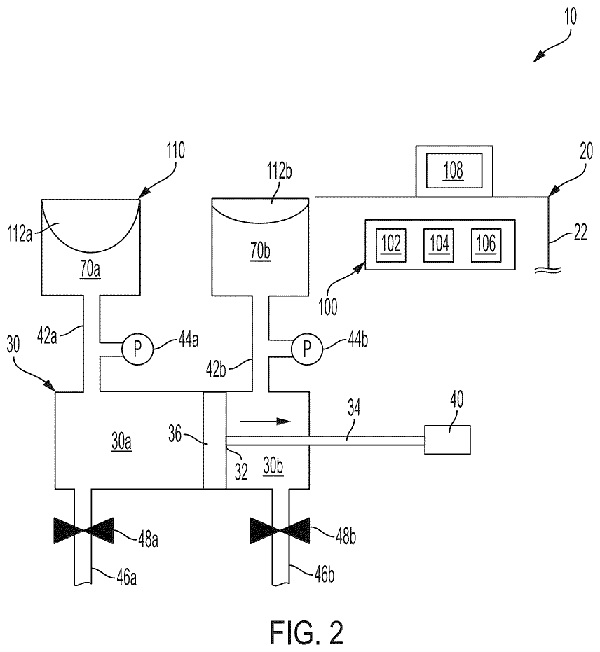

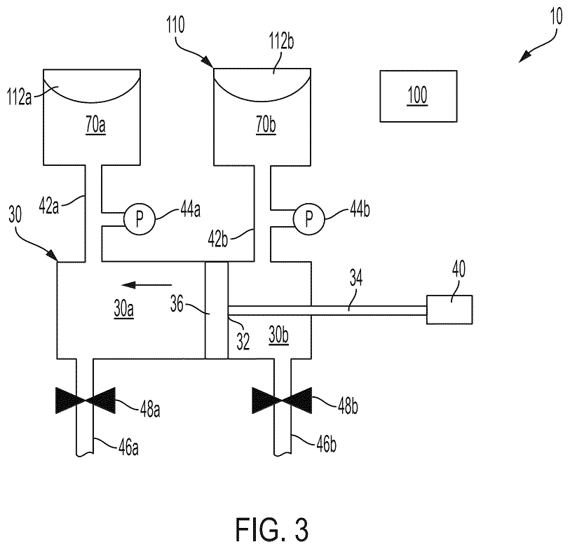

In a first primary embodiment for system 10 as illustrated in FIGS. 2 and 3, an air cylinder 30 is provided. Air cylinder 30 resides between first and second pneumatic pump chambers 70a, 70b, which operate with fluid pump chamber 112a, 112b, respectively, of disposable set 110. A piston 32 is located within air cylinder 30, wherein piston 32 includes a piston shaft 34 and a piston head 36 separating air cylinder 30 into a first cylinder chamber 30a and a second cylinder chamber 30b. Piston shaft 34 and piston head 36 in the illustrated embodiment are driven by a linear actuator 40. Linear actuator 40 may include a motor, such as a stepper motor that drives a rotational to translation conversion device such as lead or ball screw. Linear actuator 40 may alternatively be pneumatically driven. In any case, piston shaft 34 is coupled outside air cylinder 30 to linear actuator 40 for translating the piston shaft and piston head 36 within the cylinder.

A first pneumatic line 42a extends from first cylinder chamber 30a to first pneumatic pump chamber 70a. A second pneumatic 42b line extends from second cylinder chamber 30b to second pneumatic pump chamber 70b. A first pressure sensor 44a is located so as to read air pressure in first pneumatic line 42a, first pneumatic pump chamber 70 a and first cylinder chamber 30a. A second pressure sensor 44b is located so as to read air pressure in second pneumatic line 42b, second pneumatic pump chamber 70b and second cylinder chamber 30b.

A first vent line 46a and associated first vent valve 48 a are optionally placed in fluid communication with first cylinder chamber 30a. A second vent line 46b and associated second vent valve 48b are optionally placed in fluid communication with second cylinder chamber 30b.

All fluid valve actuators (driving fluid valve seats 118a to 118g), the motor or other driver of linear actuator 40, PD fluid heater 24 and other controllable electrical devices are under control of control unit 100, which includes at least one processor 102, at least one memory 104 and a video controller 106 for controlling a user interface 108 (which may be coupled to cycler 20 as illustrated or be a wireless user interface). The control unit is further configured to receive signals from all sensors, such as all pneumatic pressure sensors (e.g., 44a, 44b), fluid pressure sensors (if provided), a motor encoder (or other location determining mechanism for linear actuator 40), and any temperature sensors associated with heater 24. Control unit 100 may also include a transceiver (not illustrated) and a wired or wireless connection to a network, e.g., the internet, for sending treatment data to and receiving prescription instructions from a doctor's or clinician's server interfacing with a doctor's or clinician's computer. User interface 108 may include a display screen operating with a touchscreen and/or one or more electromechanical button, such as a membrane switch. User interface 108 may also include one or more speaker for outputting alarms, alerts and/or voice guidance commands.

To achieve the desired fresh or used PD fluid pumping sequences described herein, control unit 100 may feed the difference between a commanded pressure and a pressure measured at a relevant pressure sensor, e.g., pressure sensor 44a, 44b into a control algorithm, e.g., a proportional, integral, differential (“PID”) algorithm, which attempts to reduce the difference between the commanded pressure and the measured pressure to zero, and which results in an output to the electronic motor driver in one example of linear actuator 40. The control algorithm analysis is performed on some periodic frequency in each of the primary embodiments of system 10 described herein. Control unit 100 is programmed to run all pumping sequences discussed herein, including the sequence of the first primary embodiment discussed next.

As illustrated in FIGS. 2 and 3, PD machine or cycler 20 operates disposable set 110. Disposable set 110 among other items includes first and second fluid pump chambers 112a, 112b that operate respectively with the first and second pneumatic pump chambers 70a, 70b. When piston head 36 is moved so as to create a negative pneumatic pressure within the first or second cylinder chambers 30a, 30b, a corresponding negative pressure is created in the respective first or second pneumatic pump chambers 70a, 70b. The negative pressure created in the first or second pneumatic pump chamber 70a, 70b in turn pulls a flexible membrane of the corresponding first or second fluid pump chamber 112a, 112b into the first or second pneumatic pump chamber 70a, 70b, such that the fluid pump chamber fills with fresh or used PD fluid. When piston head 36 is moved so as to create a positive pneumatic pressure within the first or second cylinder chambers 30a, 30b, a corresponding positive pressure is created in the respective first or second pneumatic pump chambers 70a, 70b. The positive pressure created in the first or second pneumatic pump chamber 70a, 70b in turn pushes the flexible membrane of the corresponding first or second fluid pump chamber 112a, 112b, such that the fluid pump chamber 112a, 112b closes and expels fresh or used PD fluid.

Control unit 100 in the first primary embodiment causes piston shaft 34 to translate piston head 36 back and forth within air cylinder 30, such that in one half-stroke (i) first fluid pump chamber 112a fills with fresh or used PD fluid, while second fluid pump chamber 112b expels fresh or used PD fluid. In a second half-stroke (ii) second pump chamber 112b fills with fresh or used PD fluid, while first fluid pump 112a chamber expels fresh or used PD fluid. Control unit 100 causes piston head 36 to translate back and forth in the above-described manner until a desired or prescribed volume of fresh or used PD fluid is delivered from a desired PD fluid source to a desired PD fluid destination. Fluid valves are provided and are actuated sequentially by control unit 100 to access the desired fluid source and the desired fluid destination. The fluid valves may again include magnetically actuated solenoid valves, motorized pinch valves, or pneumatically actuated valves.

In the first primary embodiment, and where patient pumping is taking place such that pressure control is important, control unit 100 monitors the outputs from first and second pressure sensors 44a, 44b, while piston head 36 is translated back and forth. Control unit 100 controls the speed of the back and forth translation such that a desirably safe negative or positive fluid pumping pressure (e.g., −1.5 psig, 1.5 to 3.0 psig) is not exceeded.

In the first primary embodiment, the amount of fresh or used PD fluid delivered to a destination is determined by maintaining a constant pressure before and after movement of piston head 36, which negates the effects of the compressibility of air within cylinder 30. Because the pressure after movement (P2) equals the pressure before movement (P1), the volume of air within cylinder 30 remains constant. Hence, the volume displaced by piston head 36 is equal to volume of fluid delivered.

### Second Primary Embodiment

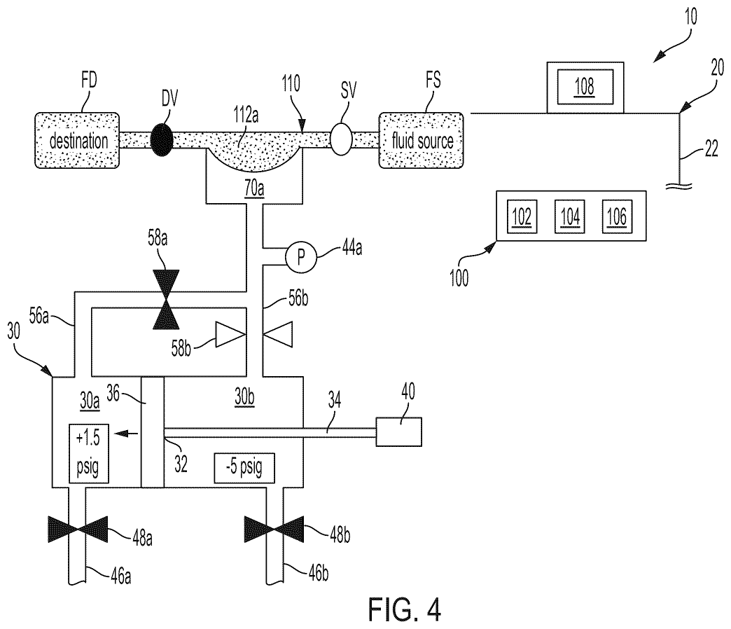

FIGS. 4 to 13 illustrate a second primary embodiment for system 10, wherein only air cylinder 30 is provided as before, but wherein the air cylinder is dedicated to a single pneumatic pump chamber 70a, 70b/fluid pump chamber pair 112a, 112b. Two pneumatic pump chamber/fluid pump chamber pairs may be provided (pair 70a, 112a is illustrated by way of example), wherein each pair has its own dedicated air cylinder 30. Air cylinder 30 is structured the same as in the first primary embodiment, including a piston 32 having a piston head 36 and piston shaft 34 driven by a linear actuator 40. Optional vent lines 46a, 46b and pneumatic vent valves 48a, 48b may be pneumatically communicated with the first and second cylinder chambers 30a, 30b of each air cylinder 30.

In the second primary embodiment, first and second pneumatic lines 56a, 56b extend from first and second cylinder chambers 30a, 30b, respectively, to the same pneumatic pump chamber 70a. First and second pneumatic valves 58a, 58b under the control of control unit 100 are provided along first and second pneumatic lines 56a, 56b. One or more pressure sensor 44a is provided along a common portion of first and second pneumatic lines 56a, 56b or along each of the first and second pneumatic lines. The fluid pump chamber(s) 112a, 112b is/are provided again as part of a disposable set 110, wherein fluid pump chamber 112a pumps fresh or used PD fluid from a desired PD fluid source FS to a desired destination FD as determined by the sequencing of a fluid source valve SV and a fluid destination valve DV.

FIG. 4 illustrates that control unit 100 in the second primary embodiment causes piston shaft 34 to translate piston head 36 towards first cylinder chamber 30a, creating positive pressure in first cylinder chamber 30a and negative pressure in second cylinder chamber 30b. Control unit 100 also causes source fluid valve SV and second pneumatic valve 58b to open, allowing negative pressure to reach pneumatic pump chamber 70a and causing the flexible membrane of fluid pump chamber 112a to be pulled into pneumatic pump chamber 70a and fill with fresh or used PD fluid.

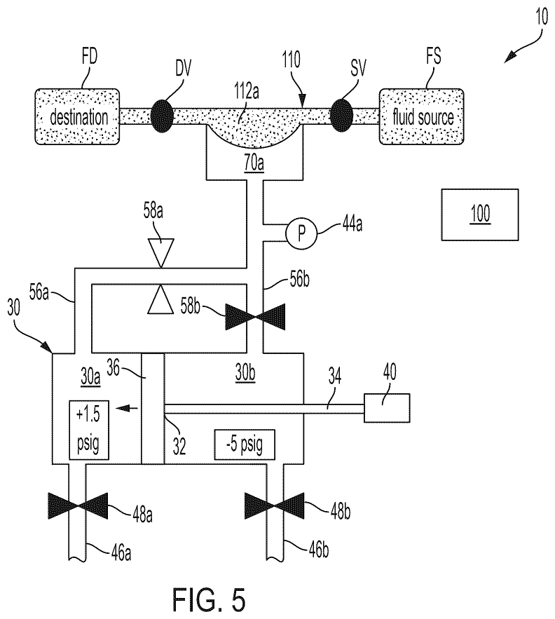

FIG. 5 illustrates that control unit 100 next causes source fluid valve SV and second pneumatic valve 58b to close and first pneumatic valve 58a to open so that pressure sensor 44a can read the positive pressure in first cylinder chamber 30a. Control unit 100 also causes piston 32 to move into first cylinder chamber 30a such that the positive pressure in pneumatic pump chamber 70a reads a desired pressure, e.g., 1.5 psig, for pumping fresh or used PD fluid to the desired destination FD. At the end of such movement, piston head 36 is at an initial piston head position.

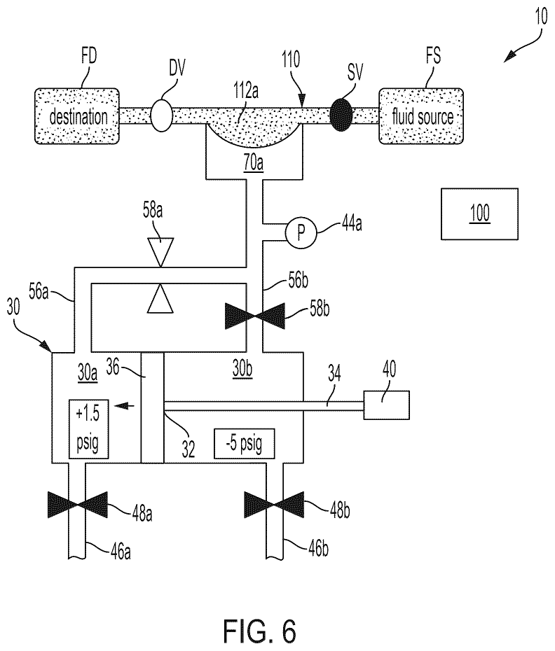

FIG. 6 illustrates that control unit 100 maintains first pneumatic valve 58a in an open state and causes destination fluid DV valve to open. The desired positive pressure built in pneumatic pump chamber 70a forces the flexible membrane of fluid pump chamber 112a to collapse and push fresh or used PD fluid to a desired destination FD. As the positive pressure dissipates, control unit 100 causes piston 32 to be moved further into first cylinder chamber 30a so that pressure sensor 44a continues to read the desired pressure, e.g., 1.5 psig.

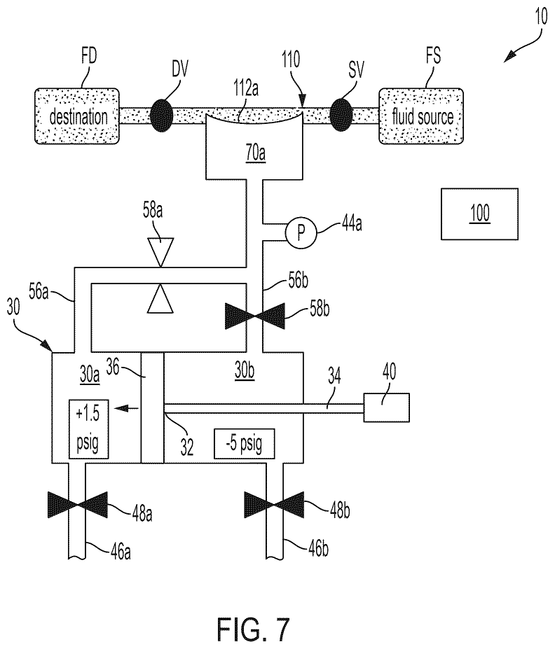

FIG. 7 illustrates that eventually, the flexible membrane cannot be collapsed any further, causing the reading at pressure sensor 44a to spike, at which time control unit 100 stops the pump-out translation of piston head 36 and closes destination fluid valve DV. The detection of the flexible membrane not being able to collapse any further may be determined alternatively or additionally by control unit 100 detecting that linear actuator 40 and/or piston head 36 is/are not moving, while the desired pressure, e.g., 1.5 psig, is maintained. In any case, after stopping the pump-out translation, first pneumatic valve 58a remains open to allow the positive pressure, which has been maintained at the desired pressure, to equalize between first cylinder chamber 30a and pneumatic pump chamber 70a, and which may be read by pressure sensor 44a. Piston head 36 is now at a final piston head position. The volume difference in the known cross-sectional area of air cylinder 30 between the final piston head position and the initial piston head position is the volume of fresh or used PD fluid pumped to desired destination FD, as determined by control unit 100, due to the pressures at the initial and final piston head positions being the same, e.g., 1.5 psig, which is a desirable pump-to-patient pressure for example. That is, a volume of space corresponding to the movement of piston head 36 within cylinder 30 is a function of a distance moved by the piston head within the cylinder and a cross-sectional area of an inner diameter of the cylinder (which basically the same as the circular area of piston head 36.

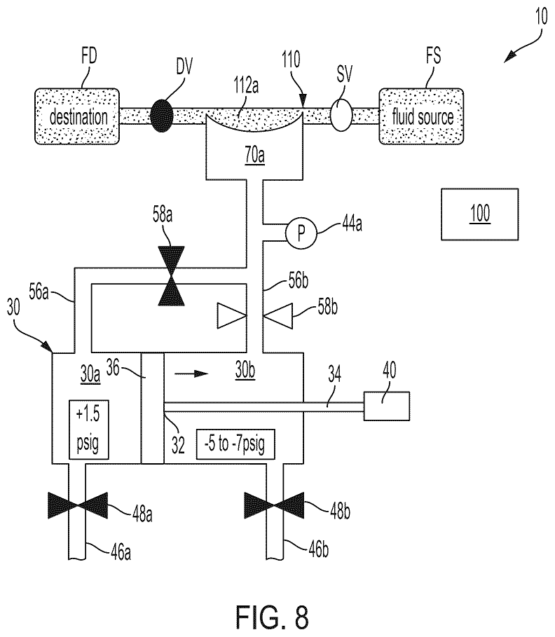

FIG. 8 illustrates that next, with second cylinder chamber 30b still being under negative pressure (which is not critical if pulling from a non-patient source), control unit 100 causes source fluid valve SV and second pneumatic valve 58b to open, allowing the negative pressure to reach pneumatic pump chamber 70a and causing the flexible membrane of fluid pump chamber 112a to be pulled into pneumatic pump chamber 70a and fill with fresh or used PD fluid.

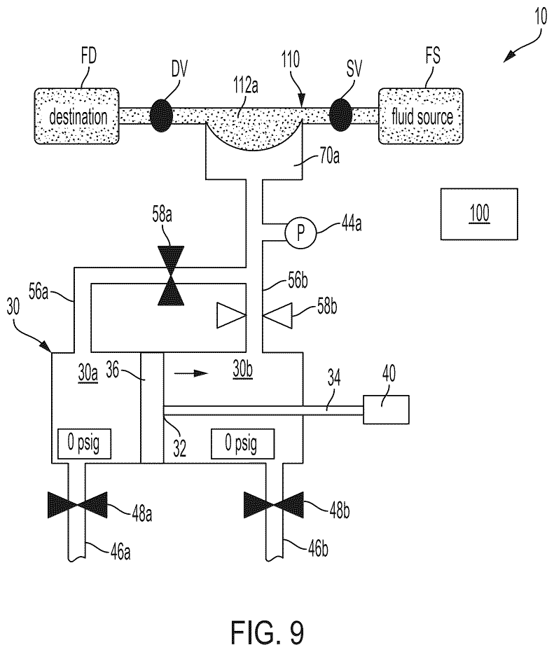

FIG. 9 illustrates that control unit 100 next causes source fluid valve SV to close but allows the second pneumatic valve 58b to remain open, such that pneumatic pump chamber 70a and second cylinder chamber 30b remain exposed to pressure sensor 44a. Control unit 100 causes piston 32 to be translated into second cylinder chamber 30b until pressure sensor 44a reads zero psig. The pressure in first cylinder chamber 30a should also be close to zero psig.

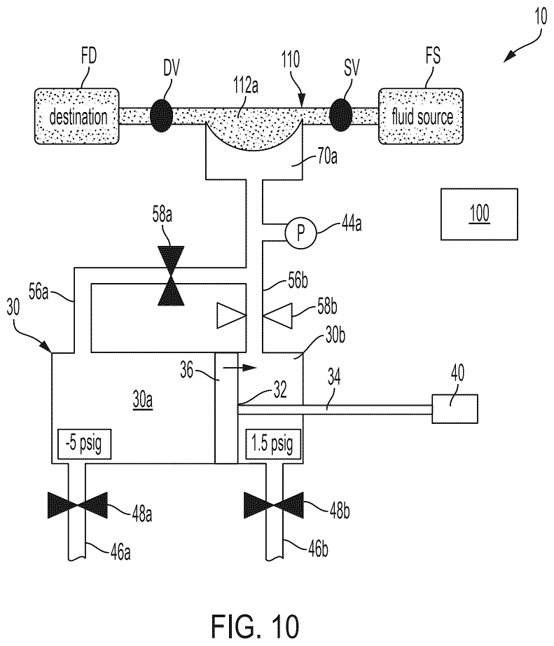

FIG. 10 illustrates that control unit 100 next with the source and destination fluid valves SV, DV closed, first pneumatic valve 58a closed, and second pneumatic valve 58b open so that pressure sensor 44a can read the positive pressure in second cylinder chamber 30b, causes piston 32 to move into second cylinder chamber 30b such that the positive pressure in pneumatic pump chamber 70a again reads a desired pressure, e.g., 1.5 psig, for pumping fresh or used PD fluid to desired destination FD. At the end of such movement, piston head 36 is at again at an initial piston head position.

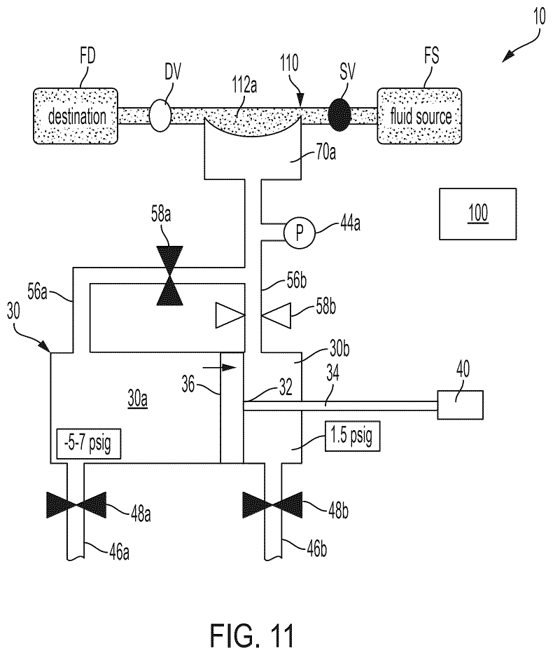

FIG. 11 illustrates that control unit 100 next maintains second pneumatic valve 58b in an open state and causes destination fluid valve DV to open. The desired positive pressure built in pneumatic pump chamber 70a again forces the flexible membrane of the fluid pump chamber 112a to collapse and push fresh or used PD fluid to desired destination FD. As the positive pressure dissipates, control unit 100 causes piston 32 to be moved further into second cylinder chamber 30b so that pressure sensor 44a continues to read the desired pressure, e.g., 1.5 psig.

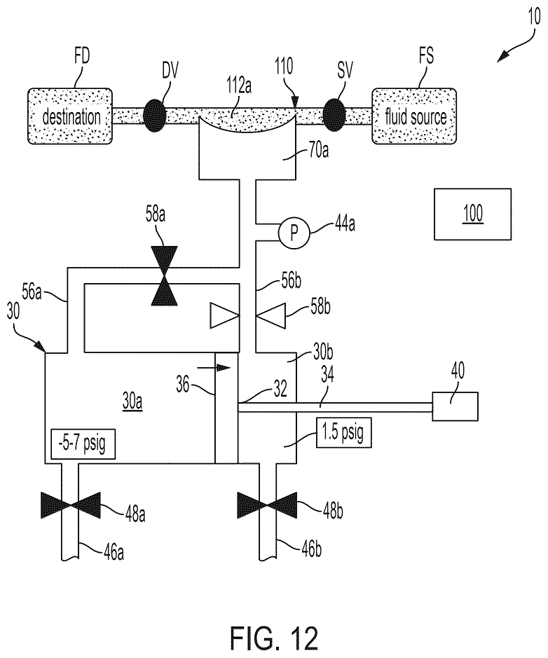

FIG. 12 illustrates that eventually, the flexible membrane cannot be collapsed any further, causing the reading at pressure sensor 44a to spike, at which time control unit 100 stops the pump-out translation of piston head 36 and closes destination fluid valve DV. The detection of the flexible membrane not being able to collapse any further may again be determined alternatively or additionally by control unit 100 detecting that linear actuator 40 and/or piston head 36 is/are not moving, while the desired pressure, e.g., 1.5 psig, is maintained. In any case, after stopping the pump-out translation, second pneumatic valve 58b remains open to allow the positive pressure, which has been maintained at the desired pressure, to equalize between first cylinder chamber 30a and pneumatic pump chamber 70a, and which may be read by pressure sensor 44a. Piston head 36 is now at a final piston head position. The volume difference in the known cross-sectional area of air cylinder 30 between the final piston head position and the initial piston head position is again the volume of fresh or used PD fluid pumped to the desired destination DV, as calculated by control unit 100, due to the pressures at the initial and final piston head positions being the same, e.g., 1.5 psig, which is a desirable pump-to-patient pressure.

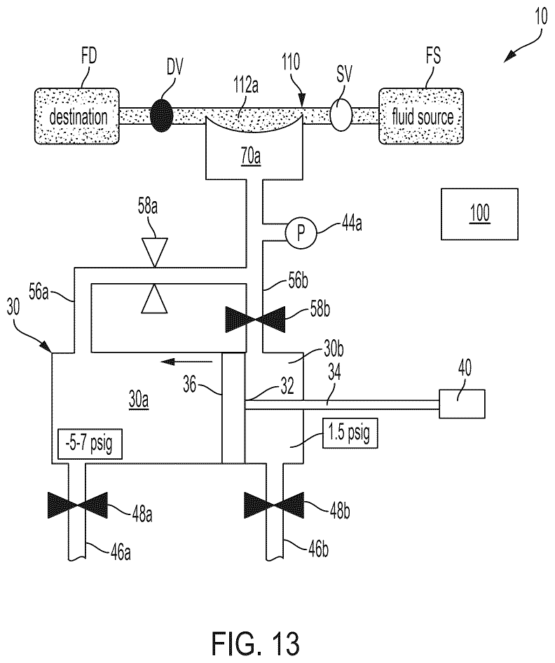

FIG. 13 illustrates that with first cylinder chamber 30a still being under negative pressure (which is not critical if pulling from a non-patient source), control unit 100 causes source fluid valve SV and first pneumatic valve 58a to open, allowing the negative pressure to reach pneumatic pump chamber 70a and causing flexible membrane of fluid pump chamber 112a to be pulled into pneumatic pump chamber 70a and fill with fresh or used PD fluid. The above process is repeated until a desired amount of fresh or used PD fluid is delivered to desired destination FD. It should be appreciated that the above process may be used for any fresh or used PD fluid source FS and any fresh or used PD fluid destination FD described herein, and that both suction and delivery pressure and PD fluid volume delivered may be controlled and measured, respectively.

### Third Primary Embodiment

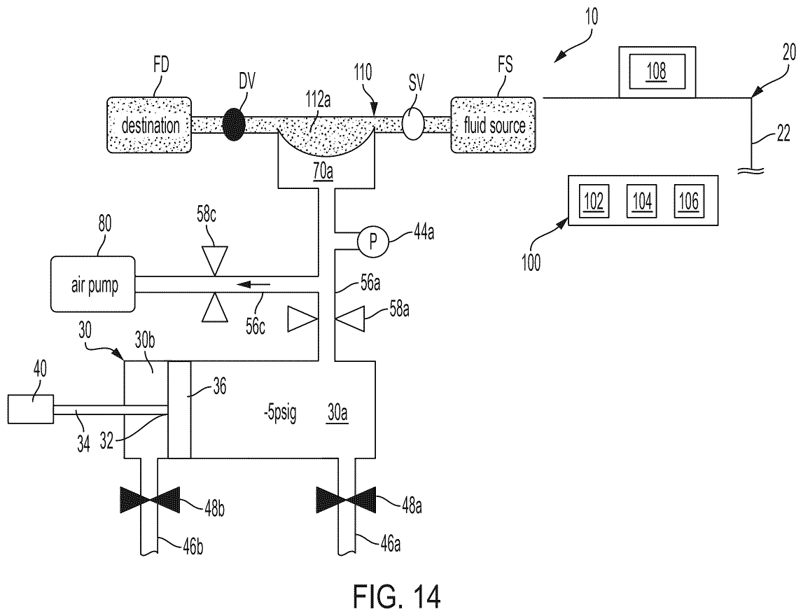

FIGS. 14 to 20 illustrate a third primary embodiment for system 10, which introduces an air pump 80 that operates in cooperation with air cylinder 30. Air pumps in general can transition more quickly between pumping positive versus negative pressure, and vice versa. Also, even small air pumps can create a wide range of pressures. Those two advantages of air pump 80 are combined with the ability of air cylinder 30 to meter a known volume of fluid under pressure control as described herein.

Air cylinder 30 in the third primary embodiment is structured roughly the same as in the first and second primary embodiments, and includes a piston head 36 and piston shaft 34 driven by a linear actuator 40. Optional vent lines 46a, 46b and pneumatic vent valves 48a, 48b may be pneumatically communicated with first and second cylinder chambers 30a, 30b, respectively, of each air cylinder 30 provided. In the third primary embodiment, only a first pneumatic line 56a extends from air cylinder 30 to pneumatic pump chamber 70a. A first pneumatic valve 58b under control of control unit 100 is provided along first pneumatic line 56a. A second pneumatic line 56c extends from air pump 80 and meets with first pneumatic line 56a. A second pneumatic valve 58v under control of control unit 100 is provided along second pneumatic line 58c. A pressure sensor 44a is provided along a common portion of the first and second pneumatic lines 56a, 56c. One or more fluid pump chamber 112a, 112b (here showing chamber 112a only by way of example) is provided again as part of a disposable set 10, wherein fluid pump chamber 112a pumps fresh or used PD fluid from a desired PD fluid source FS to a desired destination FD as determined by the sequencing of one or more fluid valve SV, DV.

FIG. 14 illustrates that control unit 100 in the third primary embodiment initially causes the first and second pneumatic valves 58a, 58c to open, the source fluid valve SV to open and air pump 80 to create a negative pressure in pneumatic pump chamber 70a and cylinder chamber 30a, pulling the flexible membrane of fluid pump chamber 112a into pneumatic pump chamber 70a and fresh or used PD fluid into fluid pump chamber 112a. In an embodiment, control unit 100 during the PD fluid draw phase monitors a speed of air pump 80. When the speed of air pump 80 begins to decrease beyond a set threshold, control unit 100 determines that the flexible membrane is fully pulled and expanded and therefore that fluid pump chamber 112a is full of fresh or used PD fluid. The speed of air pump 80 correlates directly with the flowrate of PD fluid (e.g., how fast PD fluid is loaded into pump chamber 112a). Hence, control unit 100 may be programmed to monitor the speed of air pump 80 to determine if the membrane is fully stretched (e.g., stop the air pump after a threshold change of speed is detected). Control unit 100 in an embodiment also provides closed loop control to air pump 80 so that the desired pressure is maintained. The control loop via control unit 100 may be a proportional, integral, derivative (“PID”) control loop that ensures that the pressure is not extending beyond a set threshold, which could harm the membrane.

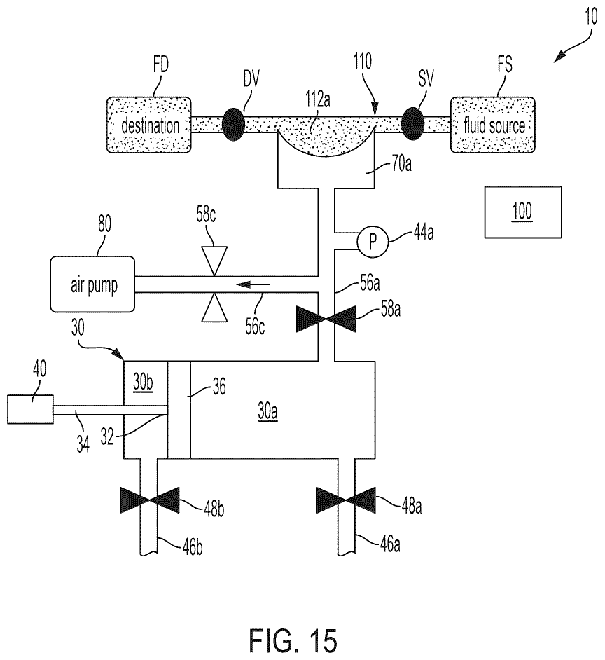

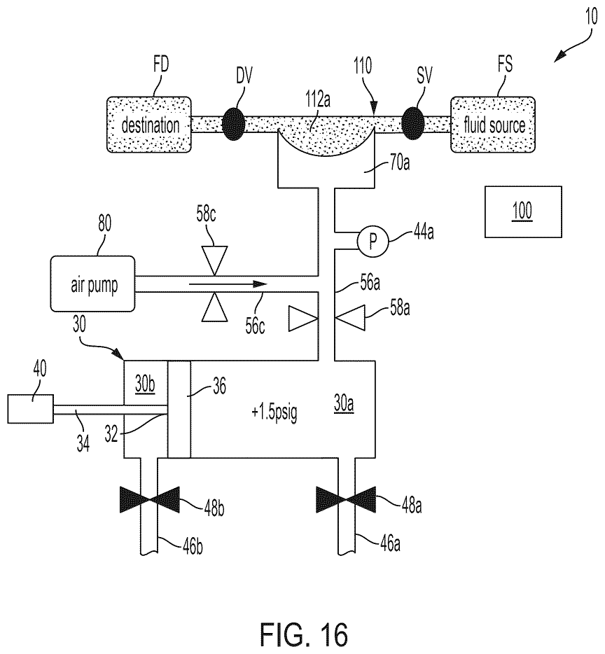

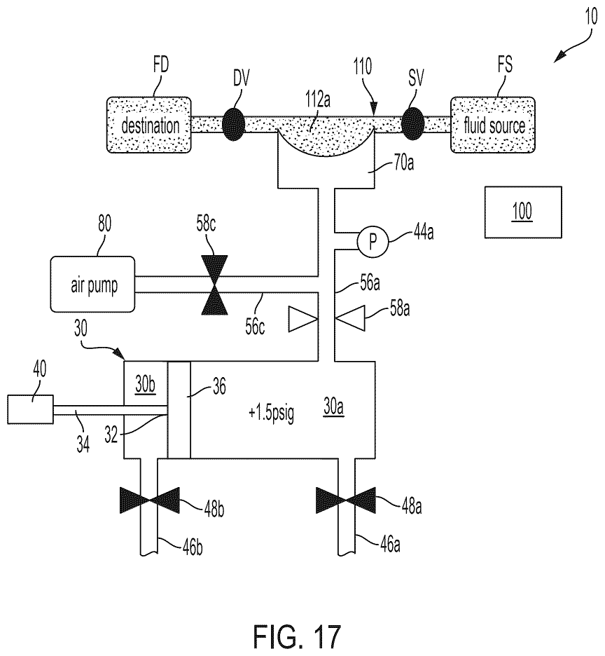

FIG. 15 illustrates that after fluid pump chamber 112a is fully filled with PD fluid, control unit 100 causes source fluid valve SV and first pneumatic valve 58a to close. FIG. 16 illustrates that control unit 100 then causes the first and second pneumatic valves 58a, 58c to open and air pump 80 to create a desired positive pumping pressure (e.g., 1.5 psig) in pneumatic pump chamber 70a and cylinder chamber 30a. FIG. 17 illustrates that once the desired positive pumping pressure is reached, control unit 100 causes second pneumatic valve 58c to close so that air pump 80 is isolated and blocked. Piston head 36 of piston 32 is here at an initial piston head position.

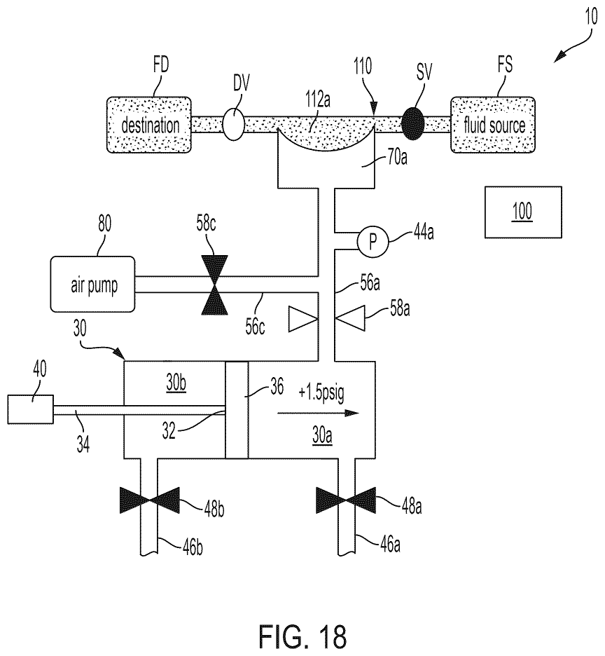

FIG. 18 illustrates that control unit 100 next maintains first pneumatic valve 58a in an open state and causes destination fluid valve DV to open. The desired positive pressure built in pneumatic pump chamber 70a forces the flexible membrane of fluid pump chamber 112a to collapse and push fresh or used PD fluid to the desired fluid destination FD. As the positive pressure dissipates, control unit 100 causes piston 32 to be moved within cylinder chamber 30a so that pressure sensor 44a continues to read the desired pressure, e.g., 1.5 psig.

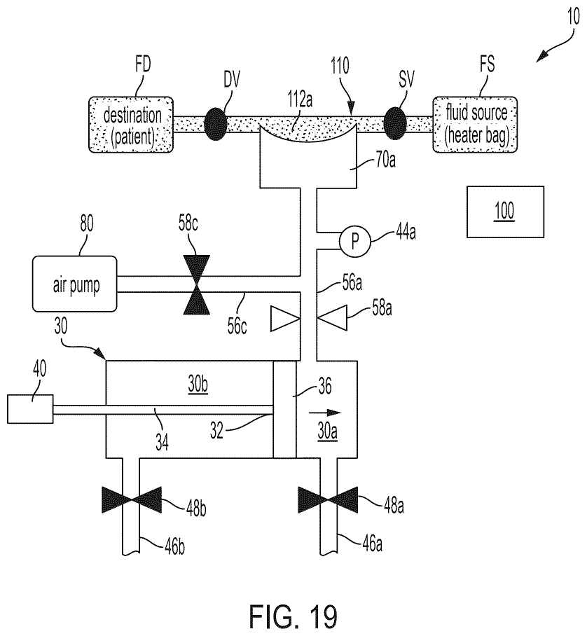

FIG. 19 illustrates that eventually, the flexible membrane of fluid pump chamber 112a cannot be collapsed any further, causing the reading of pressure sensor 44a to spike, at which time control unit 100 stops the pump-out translation of piston head 36 and closes destination fluid valve DV. The detection of the flexible membrane not being able to collapse any further may again be determined alternatively or additionally by control unit 100 detecting that linear actuator 40 and/or piston head 36 is/are not moving, while the desired pressure, e.g., 1.5 psig, is maintained. In any case, after stopping the pump-out translation, first pneumatic valve 58a remains open to allow the positive pressure, which has been maintained at the desired pressure, to equalize between cylinder chamber 30a and pneumatic pump chamber 70a, and which may be read by pressure sensor 44a. Piston head 36 is now at a final piston head position. The volume difference in the known cross-sectional area of air cylinder 30 between the final piston head position and the initial piston head position is the volume of fresh PD fluid pumped to the desired fluid destination FD, as calculated by control unit 100, due to the pressures at the initial and final piston head positions being the same, e.g., 1.5 psig, which is a desirable pump-to-patient pressure.

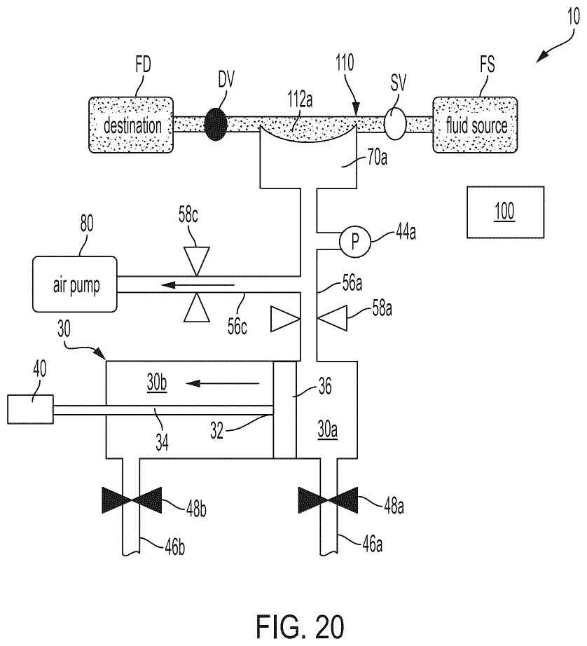

FIG. 20 illustrates that control unit 100 then causes first and second pneumatic valves 58a, 58c to open, source fluid valve SV to open and piston 32 to be moved in the opposite direction within cylinder 30 to reposition piston head 36 for the next pump-out stroke. Movement of piston 32 creates negative pressure within cylinder chamber 30a and pneumatic pump chamber 70a, which may be aided by air pump 80 to quickly achieve a desired PD fluid draw pressure. The flexible membrane of fluid pump chamber 112a is pulled into pneumatic pump chamber 70a and fresh or used PD fluid is pulled correspondingly into the fluid pump chamber. The above process for the third primary embodiment is repeated, wherein control unit 100 accumulates the pump stroke volumes, until a desired or prescribed amount of fresh or used PD fluid is delivered to the desired fluid destination FD.

### Fourth Primary Embodiment

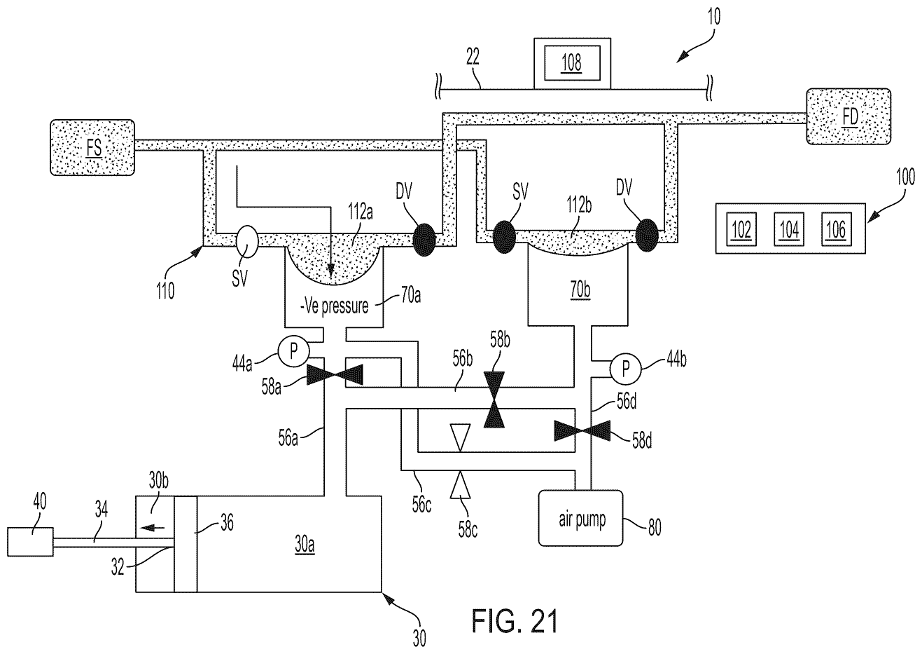

FIGS. 21 to 24 illustrate a fourth primary embodiment for system 10, which also uses air pump 80, and which operates in cooperation with air cylinder 30. Here, a single air pump 80 and air cylinder are able to drive two fluid pump chambers 112a, 112b within two pneumatic pump chambers 70a, 70b, respectively. Air cylinder 30 in the fourth primary embodiment is structured the same as in the third primary embodiment, and includes a piston head 36 and piston shaft 34 driven by a linear actuator 40. Optional vent lines and pneumatic vent valves (not illustrated) may be pneumatically communicated with the first and second cylinder chambers of each air cylinder. In the fourth primary embodiment, only a first pneumatic line 56a extends from air cylinder 30, however, first pneumatic line 56 splits to also include a second pneumatic line 56b, wherein the first and second pneumatic lines 56a, 56b extend respectively to first and second pneumatic pump chambers 112a, 112b. First and second pneumatic valves 58a, 58b under control of control unit 100 are provided along first and second pneumatic lines 56a, 56b, respectively.

A third pneumatic line 56c extends from air pump 80 and splits into a fourth pneumatic line 56d. Third pneumatic line 56c meets with first pneumatic line 56a, while fourth pneumatic line 56d meets with second pneumatic line 56b. A third pneumatic valve 58c under control of control unit 100 is provided along third pneumatic line 56c, while a fourth pneumatic valve 58d under control of control unit 100 is provided along fourth pneumatic line 56d. A first pressure sensor 44a is provided adjacent to pneumatic pump chamber 70a, while a second pressure sensor 44b is provided adjacent to pneumatic pump chamber 70b. First and second fluid pump chambers 112a, 112b are provided as part of a disposable set 100, wherein the first and second fluid pump chamber pump fresh or used PD fluid from a desired PD fluid source FS to a desired PD fluid destination FD as determined by the sequencing of a plurality of fluid valves SV, DV.

In a pumping sequence for the fourth primary embodiment, first and second fluid pump chambers 112a, 112b are generally alternated, wherein as one fluid pump chamber 112a or 112b draws fresh or used PD fluid in, the other fluid pump chamber 112b or 112a pushes fresh or used PD fluid out. Each fluid pump chamber 112a, 112b has its own set of source and destination valves SV, DV, however, it is not required that the first and second fluid pump chambers 112a, 112b are perfectly synched.

FIG. 21 illustrates that control unit 100 in the fourth primary embodiment initially causes the third pneumatic valve 58c and source fluid valve SV for first fluid pump chamber 112a to open and air pump 80 to create a negative pressure in first pneumatic pump chamber 70a, pulling the flexible membrane of first fluid pump chamber 112a into first pneumatic pump chamber 70a and fresh or used PD fluid into the first fluid pump chamber. Control unit 100 during the PD fluid draw phase may again monitor a speed of air pump 80. When the speed of air pump 80 begins to decrease beyond a threshold, control unit 100 determines that the flexible membrane is fully pulled and expanded and therefore that fluid pump chamber 112a is full of fresh or used PD fluid, at which time control unit 100 causes air pump 80 to stop and source fluid valve SV for first fluid pump chamber 112a to close.

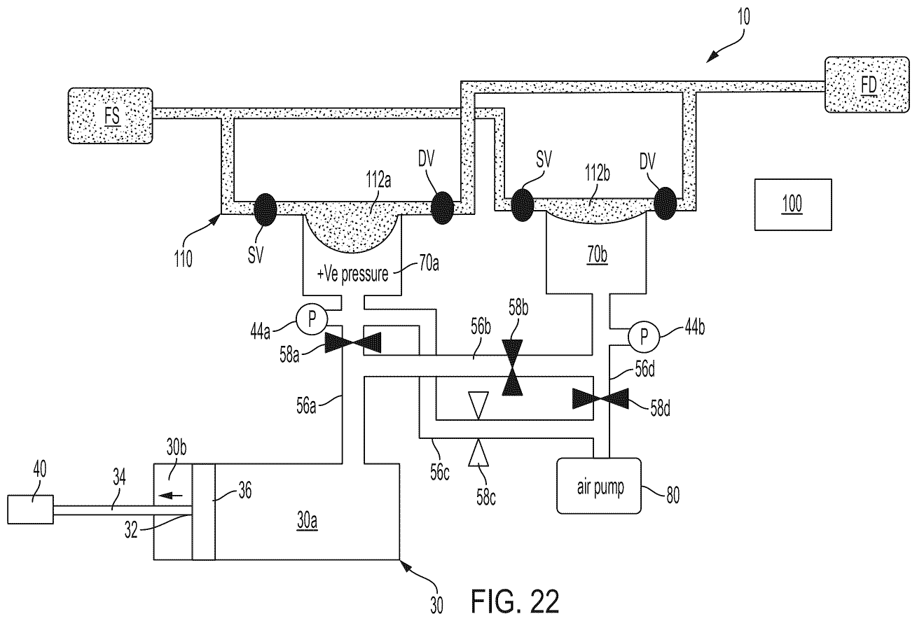

FIG. 22 illustrates that control unit 100 next maintains third pneumatic valve 58c in an open state and causes the air pump 80 to create a desired positive pumping pressure (e.g., 1.5 psig as read by the first pressure sensor 44a) in first pneumatic pump chamber 70a. At this point, piston head 36 of piston 32 of air cylinder 30 is at an initial piston head position.

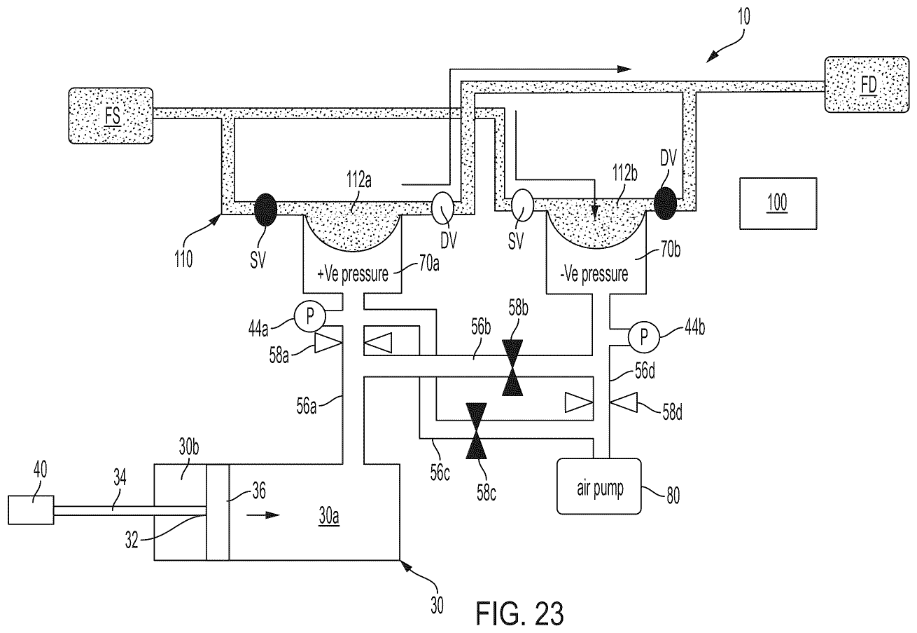

FIG. 23 illustrates that control unit 100 next causes third pneumatic valve 58c to close, first pneumatic valve 58a to open and first destination fluid valve DV to open. The desired positive pressure built in first pneumatic pump chamber 70a forces the flexible membrane of first fluid pump chamber 112a to collapse and push fresh or used PD fluid to the desired fluid destination FD. As the positive pressure dissipates, control unit 100 causes piston 36 to be moved within cylinder chamber 30a so that first pressure sensor 44a continues to read the desired pressure, e.g., 1.5 psig. Simultaneously (or near), control unit 100 causes fourth pneumatic valve 58d to open, second source valve SV to open, and air pump 80 to create a negative pressure in second pneumatic pump chamber 70b, pulling the flexible membrane of second fluid pump chamber 112b into second pneumatic pump chamber 70b and fresh or used PD fluid into the second fluid pump chamber.

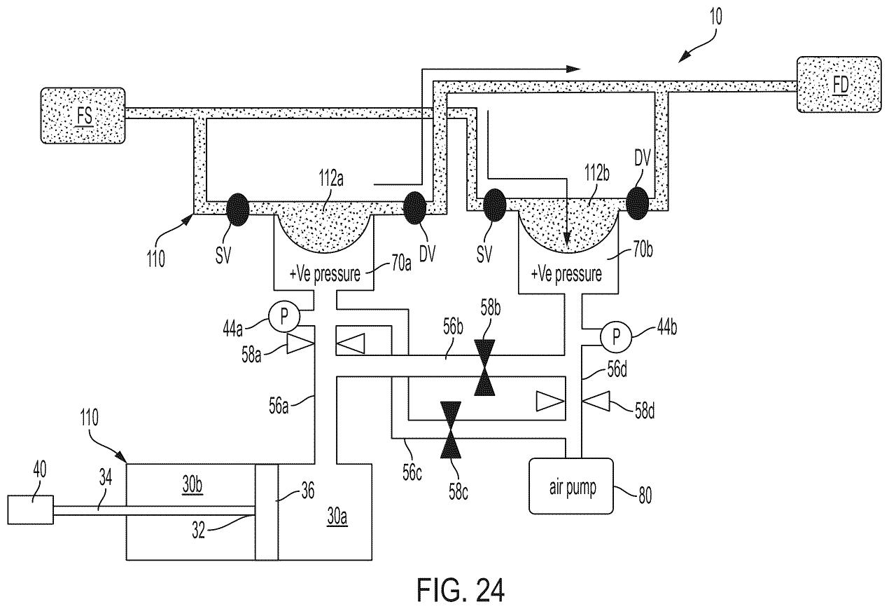

FIG. 24 illustrates that eventually, the first flexible membrane of first fluid pump chamber 112a cannot be collapsed any further, causing the reading of first pressure sensor 44a to spike, at which time control unit 100 stops the pump-out translation of piston head 36 and closes first destination fluid valve DV. The detection of the flexible membrane not being able to collapse any further may again be determined alternatively or additionally by control unit 100 detecting that linear actuator 40 and/or piston head 36 is/are not moving, while the desired pressure, e.g., 1.5 psig, is maintained. In any case, after stopping the pump-out translation, first pneumatic valve 58a remains open to allow the positive pressure, which has been maintained at the desired pressure, to equalize between cylinder chamber 30a and first pneumatic pump chamber 70a, and which may be read by first pressure sensor 44a. Piston head 36 is now at a final piston head position within cylinder chamber 30a. The volume difference in the known cross-sectional area of air cylinder 30 between the final piston head position and the initial piston head position is the volume of fresh or used PD fluid pumped to the desired fluid destination FD, as calculated by control unit 100, due to the pressures at the initial and final piston head positions being the same, e.g., 1.5 psig, which is a desirable pump-to-patient pressure. For the fluid draw of second fluid pump chamber 112b, control unit 100 may again monitor a speed of air pump 80. When the speed of air pump 80 begins to decrease beyond a threshold, control unit 100 determines that the flexible membrane of second fluid pump chamber 112b is fully pulled and expanded and therefore that the second fluid pump chamber is full of fresh or used PD fluid, at which time the control unit causes air pump 80 to stop and second source fluid valve SV for second fluid pump chamber 112b to close.

Control unit 100 then causes piston 32 to retract to an initial position and the above process to be repeated, but wherein first fluid pump chamber 112a fills with fresh or used PD fluid and the second fluid pump chamber 112b pushes fresh or used PD fluid to the desired fluid destination FD. Control unit 100 accumulates the known stroke volumes and continues to perform the alternating pumping sequence just described until a desired or prescribed amount of fresh or used PD fluid is delivered to the desired fluid destination FD.

### Fifth Primary Embodiment

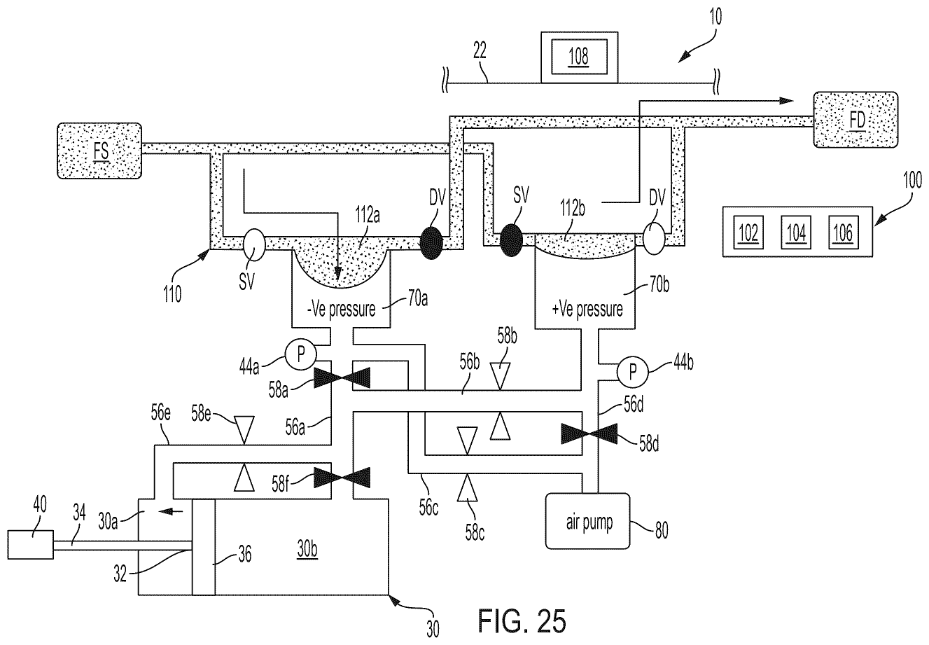

FIGS. 25 to 28 illustrate a fifth primary embodiment for system 10, which like the fourth primary embodiment, also uses an air pump 80 that operates in cooperation with air cylinder 30 to drive two pneumatic pump chambers 70a, 70b and their corresponding fluid pump chambers 112a, 112b. In the fourth primary embodiment, air cylinder 30 is unidirectional regarding fluid volume metering because only one side (chamber 30a) of piston head 36 within the cylinder is exposed to first and second pressure sensors 44a, 44b. Piston 32 needs to be reset accordingly in the fourth embodiment after each fluid pump chamber draw/fluid pump chamber deliver sequence. In the fifth primary embodiment, a fifth pneumatic line 56e is added, which extends from first pneumatic line 56a to an opposing side of air cylinder 30, so that there is pneumatic access to the air cylinder on both sides of piston head 36. A fifth pneumatic valve 58e is provided to operate with fifth pneumatic line 56e. A sixth pneumatic valve 58f is added as a second valve along first pneumatic line 56a, which allows air cylinder 30 at chamber 30b to be closed-off.

FIG. 25 illustrates that in a pumping sequence using the fifth primary embodiment of system 10, control unit 100 causes the second and fifth pneumatic valves 58b, 58f to be open, the second destination fluid valve DV to be open, and piston head 36 of air cylinder 30 to be moved in a first direction to deliver fresh or used PD fluid from second fluid pump chamber 112b to a desired destination FD. Simultaneously, control unit 100 causes third pneumatic valve 58c and the first source fluid valve SV to be open so that air pump 80 may apply a negative pressure to the flexible membrane of first fluid pump chamber 112a, pulling fresh or used PD fluid into the first fluid pump chamber.

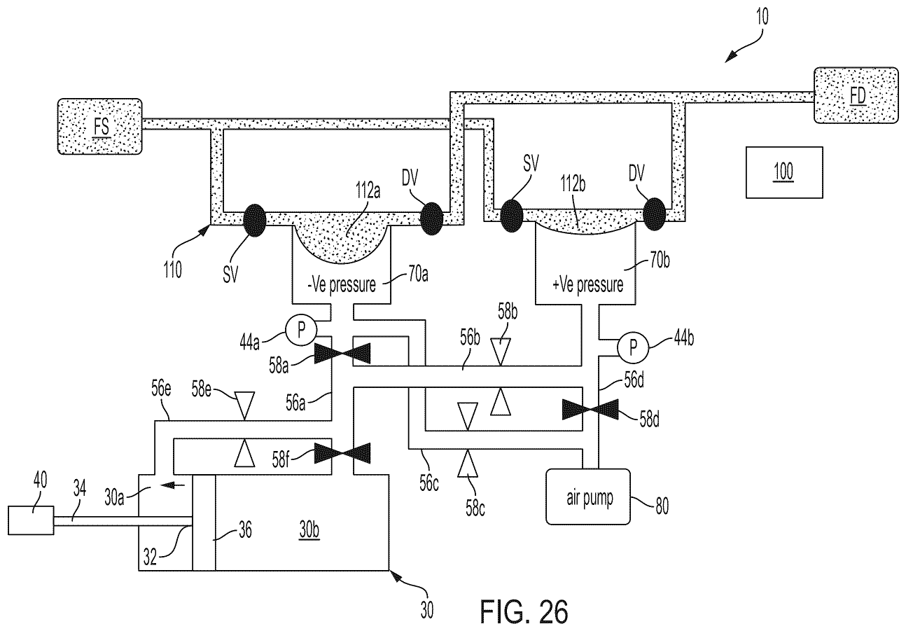

FIG. 26 illustrates that eventually, second flexible membrane second fluid pump chamber 112b cannot be collapsed any further, causing the reading of second pressure sensor 44b to spike, at which time control unit 100 stops the pump-out translation of piston head 36 and closes second destination fluid valve DV. The detection of the flexible membrane not being able to collapse any further may again be determined alternatively or additionally by control unit 100 detecting that linear actuator 40 and/or piston head 36 is/are not moving, while the desired pressure, e.g., 1.5 psig, is maintained. In any case, after stopping the pump-out translation, second and fifth pneumatic valves 58b, 58e remain open to allow the positive pressure, which has been maintained at the desired pressure, to equalize between cylinder chamber 30a and second pneumatic pump chamber 70b, and which may be read by second pressure sensor 44b. The volume difference in the known cross-sectional area of air cylinder 30 between the initial and final piston head positions is the volume of fresh or used PD fluid pumped to the desired fluid destination DV, as calculated by control unit 100, due to the pressures at the initial and final piston head positions being the same, e.g., 1.5 psig, which is a desirable pump-to-patient pressure. For the fluid draw of first fluid pump chamber 112a, control unit 100 may again monitor a speed of air pump 80. When the speed of air pump 80 begins to decrease beyond a threshold, control unit 100 determines that the flexible membrane of first fluid pump chamber 122a is fully pulled and expanded and therefore that the first fluid pump chamber is full of fresh or used PD fluid, at which time control unit 100 causes air pump 80 to stop and first source fluid valve SV for first fluid pump chamber 112a to close.

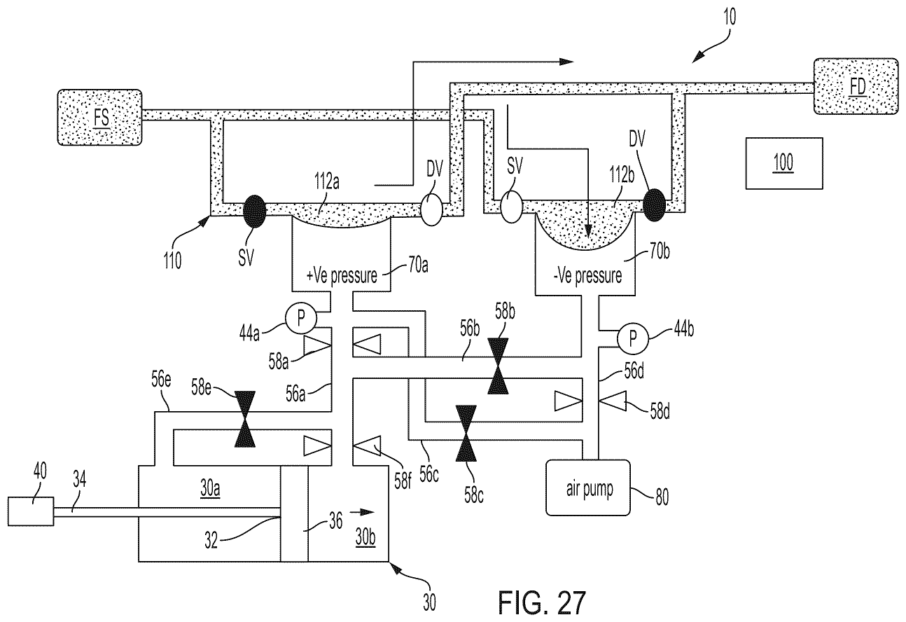

FIG. 27 illustrates that next, first and second fluid pump chambers112a, 112b switch operation so that second fluid pump chamber 112b draws fresh or used PD fluid in, while first fluid pump chamber 112a delivers the fresh or used PD fluid. Notably, no adjustment of piston head 36 is needed to ready air cylinder 30 for the switch. Here, control unit 100 causes first and sixth pneumatic valves 58a, 58f to be open, first destination fluid valve DV to be open and piston head 36 of air cylinder 30 to be moved in a second direction to deliver fresh or used PD fluid from first fluid pump chamber 112a to a desired fluid destination FD. Simultaneously, control unit 100 causes fourth pneumatic valve 58d and second source fluid valve SV to be open so that air pump 80 may apply a negative pressure to the flexible membrane of second fluid pump chamber 112b, pulling fresh or used PD fluid into the second fluid pump chamber.

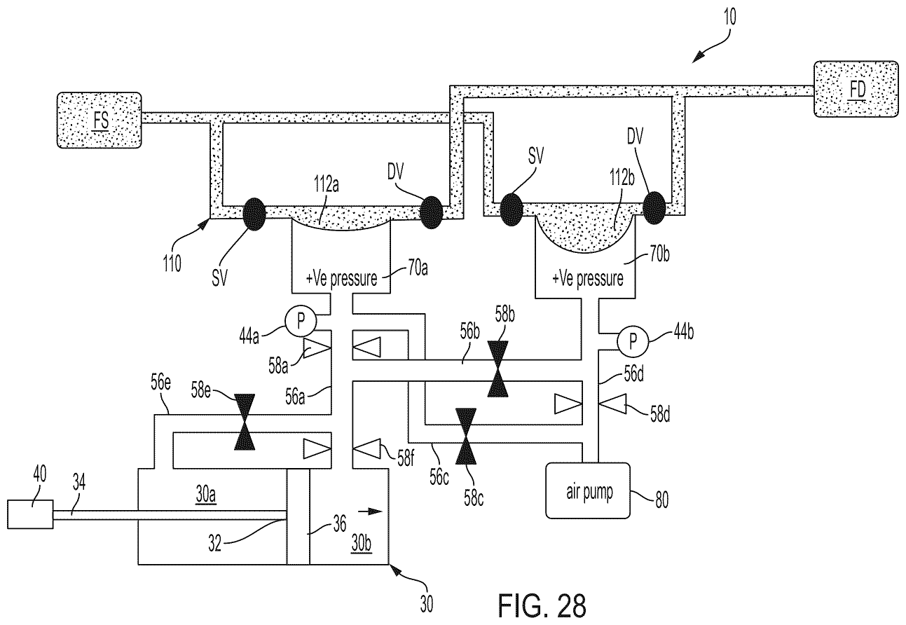

FIG. 28 illustrates that eventually, first flexible membrane of first fluid pump chamber 112a cannot be collapsed any further, causing the reading at first pressure sensor 44a to spike, at which time control unit 100 stops the pump-out translation of piston head 36 and closes first destination fluid valve DV. The detection of the flexible membrane not being able to collapse any further may again be determined alternatively or additionally by control unit 100 detecting that linear actuator 40 and/or piston head 36 is/are not moving, while the desired pressure, e.g., 1.5 psig, is maintained. In any case, after stopping the pump-out translation, the first and sixth pneumatic valves 58a, 58f remain open to allow the positive pressure, which has been maintained at the desired pressure, to equalize between second cylinder chamber 30b and first pneumatic pump chamber 70a, and which may be read by first pressure sensor 44a. The volume difference in the known cross-sectional area of air cylinder 30 between the initial and final piston head positions is the volume of fresh or used PD fluid pumped to the desired fluid destination FD, as calculated by control unit 100, due to the pressures at the initial and final piston head positions being the same, e.g., 1.5 psig, which is a desirable pump-to-patient pressure. For the fluid draw of second fluid pump chamber 112b, control unit 100 may again monitor a speed of air pump 80. When the speed of air pump 80 begins to decrease beyond a threshold, control unit 100 determines that the flexible membrane of the second fluid pump chamber 112b is fully pulled and expanded and therefore that the second fluid pump chamber is full of fresh or used PD fluid, at which time control unit 100 causes air pump 80 to stop and the second source fluid valve SV for second fluid pump chamber 112b to close.

Control unit 100 accumulates the known stroke volumes and continues to perform the alternating pumping sequence just described until a desired or prescribed amount of fresh or used PD fluid is delivered to the desired fluid destination FD. It should be appreciated for any of the above primary embodiments one to five that for fresh or used PD fluid destinations that do not involve the patient (e.g., heater container 116a or drain container 116b), the delivery pressure may be higher, e.g., up to eight psig. It is also contemplated for any of the primary embodiments one to five that control unit 100 monitors the relevant first or second pressure sensor 44a, 44b when either air cylinder 30 or air pump 80 is removing used PD fluid from the patient, so that a patient drain negative pressure limit, e.g., −1.5 psig, is not met or is not exceeded.

It should be appreciated that the effects of drift in the pneumatic pressure sensors 44a, 44b for the above embodiments are negated because the important aspect for the above sequences involving air cylinder 30 is that the initial and final pressures associated with fresh or used PD fluid delivery are equal, and not that the pressures are accurate from an absolute standpoint (except for patient pumping pressure limits). Also, because system 10 is pressure controlled, linear actuator 40 does not have to be highly accurate.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A peritoneal dialysis system comprising:
a pneumatic pump chamber;
a cylinder;
a piston including a piston head slideably sealed within the cylinder, the piston head separating a first cylinder chamber from a second cylinder chamber;
a linear actuator in mechanical communication with the piston;
a first pneumatic line extending between the first cylinder chamber and the pneumatic pump chamber;
a second pneumatic line extending between the second cylinder chamber and the pneumatic pump chamber;
a first pneumatic valve located along the first pneumatic line;
a second pneumatic valve located along the second pneumatic line;
a pressure sensor positioned and arranged to measure a pressure in the pneumatic pump chamber;
a fluid pump chamber operably coupled with the pneumatic pump chamber;
a source fluid valve;
a destination fluid valve; and
a control unit configured to cause (i) the second pneumatic valve and the source fluid valve to open and the linear actuator to move the piston head into the first cylinder chamber so as to create a negative pneumatic pressure in the second cylinder chamber and the pneumatic pump chamber, pulling source fluid into the fluid pump chamber and (ii) the second pneumatic valve and the source fluid valve to close, the first pneumatic valve and the destination fluid valve to open and the linear actuator to move the piston head further into the first cylinder chamber so as to push source fluid through the destination fluid valve, and wherein the control unit uses an output from the pressure sensor to control the linear actuator so that a final pressure for (ii) at least substantially equals an initial pressure for (ii), such that a volume of space corresponding to the movement of the piston head within the cylinder during (ii) equals a volume of the source fluid delivered from fluid pump chamber during (ii).

2. The peritoneal dialysis system of claim 1, wherein the source fluid valve is for a PD fluid supply container, a heating container, or a patient line.

3. The peritoneal dialysis system of claim 1, wherein the destination fluid valve is for a heating container, a drain container, or a patient line.

4. The peritoneal dialysis system of claim 1, wherein the volume of space corresponding to the movement of the piston head within the cylinder is a function of a distance moved by the piston head within the cylinder and a cross-sectional area of an inner diameter of the cylinder.

5. The peritoneal dialysis system of claim 1, wherein the control unit is configured to determine the volume of the source fluid delivered from fluid pump chamber during (ii) with both the source fluid valve and the destination fluid valve closed.

6. The peritoneal dialysis system of claim 1, wherein the control unit is further configured to cause, with the first pneumatic valve and the destination fluid valve closed, (iii) the second pneumatic valve and the source fluid valve to open to allow the negative pneumatic pressure in the second cylinder chamber created during at least one of (i) or (ii) to reach the pneumatic pump chamber, pulling source fluid into the fluid pump chamber.

7. The peritoneal dialysis system of claim 6, wherein the control unit is further configured to cause, with the first pneumatic valve, the source fluid valve and the destination fluid valve closed and the second pneumatic valve closed, (iv) the linear actuator to move the piston head into the second cylinder chamber so as to create at least substantially zero pressure in the first and second cylinder chambers.

8. The peritoneal dialysis system of claim 7, wherein the control unit is further configured to cause (v) the second pneumatic valve and the destination fluid valve to open and the linear actuator to move the piston head into the second cylinder chamber so as to push source fluid through the destination fluid valve, and wherein the control unit uses an output from the pressure sensor to control the linear actuator so that a final pressure for (v) at least substantially equals an initial pressure for (v), such that a volume of space corresponding to the movement of the piston head within the cylinder during (v) equals a volume of the source fluid delivered from fluid pump chamber during (v).

9. The peritoneal dialysis system of claim 8, wherein the control unit is further configured to cause, with the second pneumatic valve and the destination fluid valve closed, (vi) the first pneumatic valve and the source fluid valve to open to allow the negative pneumatic pressure in the first cylinder chamber created during (v) to reach the pneumatic pump chamber, pulling source fluid into the fluid pump chamber.

10. A peritoneal dialysis system comprising:
a pneumatic pump chamber;
a cylinder;
a piston including a piston head slideably sealed within the cylinder;
a linear actuator in mechanical communication with the piston;
an air pump;
a first pneumatic line extending between the cylinder and the pneumatic pump chamber;
a second pneumatic line extending between the air pump and the pneumatic pump chamber;
a first pneumatic valve located along the first pneumatic line;
a second pneumatic valve located along the second pneumatic line;
a pressure sensor positioned and arranged to measure a pressure in the pneumatic pump chamber;
a fluid pump chamber operably coupled with the pneumatic pump chamber;
a source fluid valve;
a destination fluid valve; and
a control unit configured to cause (i) the second pneumatic valve and the source fluid valve to open and the air pump to create a negative pneumatic pressure in the pneumatic pump chamber, pulling source fluid into the fluid pump chamber, (ii) the source fluid valve to close and with the second pneumatic valve open, the air pump to create a desired positive pressure, as measured by the pressure sensor, in the pneumatic pump chamber, and (iii) the second pneumatic valve to close, the first pneumatic valve and the destination fluid valve to open, and the linear actuator to move the piston head within the cylinder so as to push source fluid through the destination fluid valve, and wherein the control unit uses an output from the pressure sensor to control the linear actuator so that a final pressure for (iii) at least substantially equals an initial pressure for (iii), such that a volume of space corresponding to the movement of the piston head within the cylinder during (iii) equals a volume of the source fluid delivered from fluid pump chamber during (iii).

11. The peritoneal dialysis system of claim 10, wherein the source fluid valve is for a PD fluid supply container, a heating container, or a patient line.

12. The peritoneal dialysis system of claim 10, wherein the destination fluid valve is for a heating container, a drain container, or a patient line.

13. The peritoneal dialysis system of claim 10, wherein the volume of space corresponding to the movement of the piston head within the cylinder is a function of a distance moved by the piston head within the cylinder and a cross-sectional area of an inner diameter of the cylinder.

14. The peritoneal dialysis system of claim 10, wherein during (i) the first pneumatic valve is open.

15. The peritoneal dialysis system of claim 10, wherein the control unit is further configured to cause, with the first pneumatic valve and the source fluid valve open, (iv) the linear actuator to move the piston head within the cylinder in an opposite direction, pulling source fluid into the fluid pump chamber.

16. The peritoneal dialysis system of claim 15, wherein during (iv) the first pneumatic valve is open and the air pump is actuated to aid in pulling source fluid into the fluid pump chamber.

17. A peritoneal dialysis system comprising:
a first pneumatic pump chamber;
a second pneumatic pump chamber;
a cylinder;
a piston including a piston head slideably sealed within the cylinder;
a linear actuator in mechanical communication with the piston;
an air pump;
a first pneumatic line extending between the cylinder and the first pneumatic pump chamber;
a second pneumatic line extending between the cylinder and the second pneumatic pump chamber;
a third pneumatic line extending between the air pump and the first pneumatic pump chamber;
a fourth pneumatic line extending between the air pump and the second pneumatic pump chamber;
a first pneumatic valve located along the first pneumatic line;
a second pneumatic valve located along the second pneumatic line;
a third pneumatic valve located along third first pneumatic line;
a fourth pneumatic valve located along the fourth pneumatic line;
a first pressure sensor positioned and arranged to measure a pressure in the first pneumatic pump chamber;
a second pressure sensor positioned and arranged to measure a pressure in the second pneumatic pump chamber;
a first fluid pump chamber operably coupled with the first pneumatic pump chamber;
a first source fluid valve for the first pump chamber;
a first destination fluid valve for the first pump chamber;
a second fluid pump chamber operably coupled with the first pneumatic pump chamber;
a second source fluid valve for the second pump chamber;
a second destination fluid valve for the second pump chamber; and
a control unit configured to use the air pump to create negative and positive pneumatic pressures in the first and second pneumatic pump chambers, and to actuate the linear actuator to move the piston head within the cylinder while equalizing initial and final positive pneumatic pressures to meter determinable volumes of source fluid through the first and second destination fluid valves.

18. The peritoneal dialysis system of claim 17, wherein the control unit is configured to cause:
(i) with the third pneumatic valve and the first source fluid valve open, the air pump to create a negative pneumatic pressure in the first pneumatic pump chamber, pulling source fluid into the first fluid pump chamber,
(ii) with the third pneumatic valve open and the first source fluid valve closed, the air pump to create a desired positive pneumatic pressure in the first pneumatic pump chamber as measured by the first pressure sensor, and
(iii) with the first pneumatic valve and the first destination fluid valve open, the linear actuator to move the piston head within the cylinder so as to push source fluid through the first destination fluid valve, and wherein the control unit uses an output from the first pressure sensor to control the linear actuator so that a final pressure for (iii) at least substantially equals an initial pressure for (iii), such that a volume of space corresponding to the movement of the piston head within the cylinder during (iii) equals a volume of the source fluid delivered from the first fluid pump chamber during (iii), and with the fourth pneumatic valve and the second source fluid valve open, the air pump to create a negative pneumatic pressure in the second pneumatic pump chamber, pulling source fluid into the second fluid pump chamber.

19. The peritoneal dialysis system of claim 18, wherein the control unit is further configured to cause, with the piston head moved to a retracted position:
(iv) with the fourth pneumatic valve open and the second source fluid valve closed, the air pump to create a desired positive pneumatic pressure in the second pneumatic pump chamber as measured by the first pressure sensor, and
(v) with the second pneumatic valve and the second destination fluid valve open, the linear actuator to move the piston head within the cylinder so as to push source fluid through the second destination fluid valve, and wherein the control unit uses an output from the second pressure sensor to control the linear actuator so that a final pressure for (v) at least substantially equals an initial pressure for (v), such that a volume of space corresponding to the movement of the piston head within the cylinder during (v) equals a volume of the source fluid delivered from the second fluid pump chamber during (v), and with the third pneumatic valve and the first source fluid valve open, the air pump to create a negative pneumatic pressure in the first pneumatic pump chamber, pulling source fluid into the first fluid pump chamber.

20. A peritoneal dialysis system comprising:
a first pneumatic pump chamber;
a second pneumatic pump chamber;
a cylinder;
a piston including a piston head slideably sealed within the cylinder, the piston head separating a first cylinder chamber from a second cylinder chamber;
a linear actuator in mechanical communication with the piston;
an air pump;
a first pneumatic line extending between the second cylinder chamber and the first pneumatic pump chamber;
a second pneumatic line extending between the second cylinder chamber and the second pneumatic pump chamber;
a third pneumatic line extending between the air pump and the first pneumatic pump chamber;
a fourth pneumatic line extending between the air pump and the second pneumatic pump chamber;
a fifth pneumatic line extending between the first cylinder chamber and the first pneumatic line;
a first pneumatic valve located along the first pneumatic line;
a second pneumatic valve located along the second pneumatic line;
a third pneumatic valve located along third first pneumatic line;
a fourth pneumatic valve located along the fourth pneumatic line;
a fifth pneumatic valve located along the fifth pneumatic line;
a sixth pneumatic valve located adjacent to the second cylinder chamber;
a first pressure sensor positioned and arranged to measure a pressure in the first pneumatic pump chamber;
a second pressure sensor positioned and arranged to measure a pressure in the second pneumatic pump chamber;
a first fluid pump chamber operably coupled with the first pneumatic pump chamber;
a first source fluid valve for the first pump chamber;
a first destination fluid valve for the first pump chamber;
a second fluid pump chamber operably coupled with the first pneumatic pump chamber;
a second source fluid valve for the second pump chamber;
a second destination fluid valve for the second pump chamber; and
a control unit configured to use the air pump to create negative and positive pneumatic pressures in the first and second pneumatic pump chambers, and to actuate the linear actuator to move the piston head within the first and second cylinder chambers while equalizing initial and final positive pneumatic pressures to meter determinable volumes of source fluid through the first and second destination fluid valves.

21. The peritoneal dialysis system of claim 20, wherein the control unit is configured to cause:
(i) with the third pneumatic valve and the first source fluid valve open, the air pump to create a negative pneumatic pressure in the first pneumatic pump chamber, pulling source fluid into the first fluid pump chamber, and with the second and fifth pneumatic valves and the second destination fluid valve open, the linear actuator to move the piston head towards the first cylinder chamber so as to push source fluid through the second destination fluid valve, and wherein the control unit uses an output from the second pressure sensor to control the linear actuator so that a final pressure for (i) at least substantially equals an initial pressure for (i), such that a volume of space corresponding to the movement of the piston head towards the first cylinder chamber during (i) equals a volume of the source fluid delivered from the second fluid pump chamber during (i).

22. The peritoneal dialysis system of claim 21, wherein the control unit is further configured to cause:
(ii) with the fourth pneumatic valve and the second source fluid valve open, the air pump to create a negative pneumatic pressure in the second pneumatic pump chamber, pulling source fluid into the second fluid pump chamber, and with the first and sixth pneumatic valves and the first destination fluid valve open, the linear actuator to move the piston head towards the second cylinder chamber so as to push source fluid through the first destination fluid valve, and wherein the control unit uses an output from the first pressure sensor to control the linear actuator so that a final pressure for (ii) at least substantially equals an initial pressure for (ii), such that a volume of space corresponding to the movement of the piston head towards the second cylinder chamber during (ii) equals a volume of the source fluid delivered from the second fluid pump chamber during (ii).
